(19) United States
(12) Patent Application Publication
(10) Pub. No.: US 2024/0293483 A1
(43) Pub. Date: Sep. 5, 2024

(54) PROBIOTIC COMPOSITION FOR THE TREATMENT OF INCREASED INTESTINAL PERMEABILITY

(71) Applicant: AB-BIOTICS, S.A., Sant Cugat del Valles (ES)

(72) Inventors: Marta PÉREZ GARCIA, Sant Cugat del Valles (ES); Jordi ESPADALER MAZO, Sant Cugat del Valles (ES)

(21) Appl. No.: 18/578,507

(22) PCT Filed: Jul. 13, 2022
(86) PCT No.: PCT/EP2022/069692

§ 371 Date: Jan. 11, 2024

(30) Foreign Application Priority Data
Jul. 13, 2021 (EP) 21382631.6

(51) Int. Cl. A61K 35/745; A61K 31/702; A61P 1/00; C12N 1/20; C12P 9/00

(52) U.S. Cl. CPC A61K 35/745; A61K 31/702; A61P 1/00; C12N 1/20; C12P 9/00

(57) ABSTRACT

A probiotic composition comprising Bifidobacterium longum subsp. longum CECT 7894 is provided. The probiotic composition is useful in treating, preventing, or ameliorating an intestinal barrier dysfunction (e.g., increased intestinal permeability) or associated condition, or symptoms, complications and/or sequela thereof in a subject in need thereof, by producing polyphosphate. A combination of the probiotic composition with at at least one human milk oligosaccharide is also provided.

### FIELD OF THE INVENTION

The present invention relates to the fields of medicine and microbiology and particularly, to a probiotic composition to benefit human and animal health, particularly useful in the treatment of an intestinal barrier disfunction or associated condition.

### BACKGROUND ART

Bifidobacteria are members of the human gut microbiota which play important roles in human health. In infants, gut microbiota is dominated by Bifidobacteria whereas in adulthood the levels are lower. The presence of different species of Bifidobacteria changes with age, from childhood to old age. Bifidobacteria play key roles with beneficial effects in the normal development of the gut microbiota and its barrier effect, in the absorption of dietary compounds and in the maturation of the immune system at a critical period of the first stages of life.

Reductions in Bifidobacteria are associated with higher risks of long-term disorders such as allergies, obesity or inflammatory bowel disease, which can be triggered by factors such as C-section, preterm birth, formula feeding, or pre- and post-natal antibiotic treatment. Consequently, bifidobacterial strains are being studied for their use as probiotics in the prevention and treatment of diseases.

WO2015018883A2 discloses a probiotic composition comprising Pediococcus pentosaceus CECT 8330 and optionally comprising Bifidobacterium longum CECT 7894, useful in the amelioration of excessive crying in infants. A clinical trial testing a composition of both probiotic bacteria showed that probiotic consumption caused a greater reduction in the average daily crying time and in the duration of each episode. It is also described that “From the relevant properties of the bacterial composition explained above, it is derived that the administration of the bacterial composition, it is also useful to treat other conditions characterized by gastrointestinal disturbances associated to inflammation as consequence of the immaturation of the immune system; to treat intestinal hypersensitivity and to balance excess of undesirable bacteria in the intestine”. Regarding the properties of each probiotic strain included in the composition, WO2015018883A2 discloses that P. pentosaceus CECT 8330 showed a higher capacity to induce IL-10 and consequently potentially ameliorates inflammation in the intestinal tract, whereas B. longum CECT 7894 showed a higher capacity to inhibit growth of undesirable bacteria commonly abundant in infants with excessive crying.

JP2006176450A describes a probiotic composition comprising lactic acid bacteria, such as Bifidobacterium adolescentis JCM 1251 or Bifidobacterium breve JCM 1273, capable of accumulating polyphosphoric acid by means of absorbing phosphorus. This composition may have the potential of suppressing excessive absorption of phosphorus in the small intestine and therefore have a positive effect in the prevention of various diseases including kidney stone disease.

Some strains of lactic acid bacteria and Bifidobacteria have shown the ability to produce polyphosphate (polyP), which has been discovered to have a postbiotic effect due to its role in enhancing intestinal barrier function and maintaining host intestinal homeostasis. The host-probiotic interaction is facilitated through epithelial endocytosis of probiotic-derived polyP. In the intestinal cell, polyP induces cytoprotective factors such as the heat shock protein HSP27 through integrin β1-p38 MAPK pathway.

PolyP formation capability of Bifidobacteria was suggested by Qian et al. (2011). The authors indicate that bifidobacterial strains B. adolescentis ATCC 15703 (JCM 1275), B. longum ATCC 15707, B. longum ATCC 55816, Bifidobacterium sp. BAA-718 and B. scardovii BAA-773 produce observable granules which could be consistent with polyP granules, however this has not been proved. Further, no quantification nor characterization of the granules has been performed. These granules could also be consistent with granules containing e.g., metals or protein granules. Additionally, the expression of ppk gene, which encodes polyP biosynthesis enzyme PPK, is studied in the non-probiotic strain B. scardovii BAA-773 in response to oxidative stress.

Another study also assesses the capacity of Lactobacillus, Bifidobacteria, Lactococcus and Streptococcus to form polyP through an indirect analysis which measures the amount of phosphate left in the medium after culture time (Anand et al. 2019). B. adolescentis JCM 1275 shows the highest capacity to accumulate phosphorus, however no quantification of polyP is performed in this experiment.

Furthermore, Saiki et al., 2016 indirectly quantifies polyP production by lactic acid bacteria and Bifidobacteria through the direct quantification of ATP after adding of polyphosphate kinase (PPK). PPK is the enzyme that catalyzes the reversible reaction which produces polyP and ADP from ATP and phosphate. Results show a wide variety of polyP-producing abilities between species and strains. Lacticaseibacillus paracasei subsp. paracasei JCM 1163 shows the highest polyP concentration.

These studies represent the early steps towards the use of probiotic bacteria which are able to produce polyP. Nevertheless, probiotic features are strain-dependent, even among bacteria of the same species. Therefore, it is important to find those strains able to produce significant amounts of polyP to have a beneficial effect on the host. Furthermore, they need to have a good performance in all probiotic requirements such as resistance to gastrointestinal conditions, adequate proliferation, and also be suitable for large-scale manufacture.

The abstract Xiao et al., accepted on May 30, 2022, concludes that B. longum CECT 7894 improved the efficacy of infliximab for dextran sulfate sodium (DSS)-induced colitis in mice via regulating the gut microbiota and bile acid metabolism.

### SUMMARY OF THE INVENTION

The problem to be solved by the present invention is to provide new compositions capable of having a positive effect on intestinal barrier dysfunctions in a subject in need thereof.

The inventors have found a new probiotic composition which has the ability to produce large amounts of polyphosphate (polyP) which have a positive effect on the intestinal barrier. The probiotic composition comprises a Bifidobacterium longum subsp. longum strain, a human gut origin strain adapted to human intestinal conditions. Particularly, the Bifidobacterium strain of the present invention is Bifidobacterium longum subsp. longum strain deposited under the Budapest Treaty in the Spanish Type Culture Collection (CECT) under accession number CECT 7894 (also referred in this description as KABP-042). Remarkably, besides the ability to produce large amounts of polyP, the strain of the present invention is also able to grow while producing polyP. Further, since it belongs to B. longum subsp. longum which is present in the human microbiota among all stages of life, it has the potential to have a positive effect from newborn infants to elders. Additionally, the inventors of the present invention have proven the strain to be well adapted to gastrointestinal conditions of infants and adults e.g., resistance to gastric and bile salt stress, good adherence to intestinal epithelium, utilization of complex sugars from human milk and also to have a good stability with only a 3-fold reduction over 12 months, which surprisingly differs from other Bifidobacteria known in the art.

Working examples herein provide detailed experimental data demonstrating the capacity of the probiotic composition of the present invention to produce significant amounts of polyP without compromising its proliferation rate. The continued proliferation of this strain allows the production of increasing levels of polyP, which is a postbiotic molecule with a protective effect in the intestinal barrier. Further, as understood by the skilled person in the present context, the natural habitat of this Bifidobacterium strain is the human gut. Therefore, this strain shows a clear potential to produce polyP while proliferating under these optimal environmental conditions.

EXAMPLE 1 shows that B. longum subsp. longum CECT 7894 has the highest capacity of producing polyP compared to several tested strains (e.g., B. animalis BB-12, B. adolescentis JCM 1275, L. plantarum WCFS1 and B. scardovii BAA-773). Moreover, B. longum subsp. longum CECT 7894 shows a high potential to proliferate while producing polyP, which is crucial for their colonization of the gut and can allow the proliferation of strains from early stages of life. Therefore, the early administration of this health-promoting strain in infants can be beneficial for the gut and maintain its positive effect in further stages of life.

From a long-term perspective, the fact that proliferation rate of the strain of this invention is not compromised by the high production of polyP is advantageous to subsequently obtain larger amounts of polyP in the human gut. FIG. 2 and TABLE 2 show an outstanding capacity of B. longum subsp. longum CECT 7894 to biosynthesize high amounts of polyP while proliferating at all time points considered in this study. Likewise, B. longum subsp. longum 36524™, B. longum subsp. longum ATCC 15707 and B. animalis BB-12 also produce high amounts of polyP and have high proliferation rates. However, the two B. longum strains are not able to maintain a high production of polyP at 16 hours, and B. animalis BB-12 is only able to produce detectable amounts of polyP at 16 h.

Furthermore, while B. longum subsp. longum CECT 7894 is a Human-Residential Bifidobacteria (HRB) strain, B. animalis BB-12 is classified as a non-HRB strain. HRB-strains are characterized in that they are frequently isolated from faeces and the oral cavity of healthy humans, exert better health-promoting effects and therefore serve as a better probiotic candidate for human use, since their metabolism is adapted to human gastrointestinal tract. On the contrary, B. animalis BB-12 may not adequately adapt to and colonize the human gut, resist human gut conditions and maintain its proliferation capacity while producing high amounts of polyP.

B. breve JCM 1273 shows a similar proliferation rate at 6 h, and higher proliferation rate at 16 h when compared to B. longum subsp. longum CECT 7894. Nevertheless, its capacity to produce polyP is considerably lower at both points in time.

B. adolescentis JCM 1275 is also capable of producing some amounts of polyP, however, it does not have the capacity to proliferate simultaneously. Consequently, the overall production may be compromised since the aim is to achieve a sustained presence of the strain, i.e., a sustained production of polyP. Although this strain is considered an adult-type HRB, since it is abundant in adults and elders, it is noted that it is rarely present in infants.

Notably, genomic analysis and in vitro experiments showed in EXAMPLE 3 that B. longum subsp. longum CECT 7894 has the potential to adequately adapt to the infant and adult gastrointestinal tracts. Additionally, the subspecies B. longum subsp. longum is a long-term colonizer whose prevalence and abundance in infants is higher than other strains and species, thus the strain of the invention has a high potential to colonize the baby's gut. Further, B. longum subsp. longum is also prevalent in adult and elderly human gut, thus producing beneficial effects for the host.

Additionally, although B. scardovii is known to harbor an active ppk gene and has an exceptional growth capability (as shown in EXAMPLE 1 with strain B. scardovii BAA-773), its ability to produce polyP was minimal. Further, B. scardovii is known to be a pathogenic strain, and thus it would not be appropriate for a probiotic composition.

Finally, L. plantarum WCFS1 is known to protect the intestinal barrier through polyP production, however B. longum subsp. longum CECT 7894 produces much higher amounts of polyP. In addition, L. plantarum is not a dominant group in the intestine of infants.

Overall, the strain of the present invention would be capable of producing the highest amount of polyP when administrating the same initial dose of probiotic composition to the subject. For instance, comparing tablets containing the same cfus of the different studied strains, the strain of this invention has the highest potential to produce the largest amount of polyP.

Furthermore, B. longum subsp. longum CECT 7894 in a pharmaceutical composition showed life bacteria counts are stable over time, as shown in EXAMPLE 2 and FIG. 4. These results indicate that a three-fold overdose at manufacturing would be enough to ensure 109 cfus of live bacteria at twelve months, thereby enabling a large-scale manufacturing and long-term storage of the probiotic composition.

This long-term stability of the probiotic strain B. longum subsp. longum CECT 7894 is unexpected as it is well known in the prior art that many probiotic Bifidobacteria strains have a low tolerance to oxygen and are therefore do not show an adequate stability. Although some Bifidobacteria strains such as B. pyschroaerophilum, B. indicum and B. asteroides have a higher stability, these are not HRB strains adequate for probiotic compositions. On the contrary, B. longum subsp. longum CECT 7894 is not only a HRB but also shows high stability and therefore resistance to oxygen. Thus, this strain would be suitable for the manufacture of a probiotic composition which may require long-term storage.

Furthermore, the effect of the polyP produced by B. longum CECT 7894 has been proved to have a positive effect on barrier integrity, gut permeability and gut barrier homeostasis, as shown in EXAMPLE 4. In addition, it has also been proved that such effect is related to the production of heat shock protein (HSP27) and the induction of other markers of barrier integrity including tight junction proteins; all of them induced by the presence of polyP derived from B. longum CECT 7894.

Additionally, inventors have proven in EXAMPLE 5 the ability of B. longum CECT 7894 to produce polyP in the presence of breast milk, indicating a beneficial effect of B. longum CECT 7894 in lactating infants. Breast milk contains carbohydrates HMOs. HMO Lacto-N-tetraose (LNT) is used by B. longum CECT 7894 as confirmed in EXAMPLE 3. Furthermore, LNT has been proven to positively affect the polyP biosynthesis in the B. longum CECT 7894 strain. Remarkably, EXAMPLE 6 shows that B. longum CECT 7894 is able to growth in presence of the supernatant of other Bifidobacteria able to utilize the HMO 2′-Fucosyl-lactose (2′-FL). Overall, these results demonstrate that B. longum CECT 7894 is able to growth in presence of the two most abundant HMOs in breast milk (LNT and 2′-FL) increasing the production of polyP, thus highlighting the beneficial role of B. longum CECT 7894 supplementation in e.g., infants.

Overall, it is plausibly demonstrated that B. longum CECT 7894 produces polyP in great amounts while growing, which has a positive effect in the intestinal permeability. Further, it has also been shown that the addition of HMOs positively affects the polyP biosynthesis in the B. longum CECT 7894.

The abstract Xiao et al., accepted on May 30, 2022, concludes that B. longum CECT 7894 improved the efficacy of infliximab for dextran sulfate sodium (DSS)-induced colitis via regulating the gut microbiota and bile acid metabolism. The experimental model used is DSS-induced acute colitis in mice. Ulcerative colitis is considered an inflammatory intestinal disease characterized by overt intestinal inflammation and changes in the normal gut bacteria. Treatments described in the abstract are infliximab (monoclonal antibody with immunosuppressive effect used in the treatment of inflammatory conditions such as colitis), and infliximab+B. longum CECT 7894. There is no group of animals receiving B. longum CEC 7894 alone. Infliximab is highly efficacious in treating colitis both in humans and animal models, but its use has been associated to increased risk of infection by several clinical studies (Shah et al. 2017).

The authors describe that adding B. longum CECT 7894 to infliximab changes the microbiota and bile acid metabolism. The authors acknowledge that changes in bile acids may explain the effect. B. longum CECT 7894 increased the relative abundances of genera Bifidobacterium, Blautia, Butyricicoccus, Clostridium, Coprococcus, Gemmiger, and Parabacterioides, and reduced the relative abundances of bacteria genera Enterococcus and Pseudomonas. Given that Enterococci and especially Pseudomonas can be pathogenic and that use of infliximab is known to reduce inflammation but increase the risk of infection, the mere fact of adding B. longum CECT7894 could compensate the drawbacks of infliximab therapy and thus facilitate a faster healing of the intestine by reducing the levels of pathogenic bacteria already in the intestine. Of note, the observed effect is dependent on the preexisting intestinal microbiota and on the combination with infliximab.

Besides, authors report the improvement in the DSS colitis model to be associated to changes in several bile acids. However, composition of bile acids in mice and humans is significantly different, with the former containing relevant amounts of alpha and beta murocholic acid which are virtually absent in the later, thus limiting the generalizability of the findings to humans.

On the other hand, they disclose that some parameters e.g., tight junctions (ZO-1, occludin) improve in the infliximab+B. longum CECT 7894 group, but there are no data to sufficiently evidence this effect. In summary, this abstract shows some results on the effect of B. longum CECT 7894 on the efficacy of infliximab in the specific experimental model of (DSS)-induced colitis in mouse, by regulating the gut microbiota and the bile acid metabolism.

It can not be derived an effect of B. longum CECT 7894 alone without treatment with infliximab, particularly in the experiment of tight junctions where the results, even combined to infliximab, are not conclusive. Further, in can not be derived an effect of B. longum CECT 7894 in diseases different from the experimental model of colitis used in this study since, as discussed, the observed effect, in combination with infliximab, is dependent on the preexisting intestinal microbiota.

Remarkably, the effects of B. longum CECT 7894 on ameliorating the disease (as said before, through improving the efficacy of infliximab by regulating the microbiota and the bile acid metabolism) can be considered indirect effects. Contrarily, in the present invention, a direct effect of B. longum CECT 78994 is shown, i.e., protection of the intestinal permeability through the direct delivery of polyphosphates to the intestinal epithelium. Further, the effects are independent from the disease model and the surrounding microbiota.

Altogether, inventors have found the Bifidobacterium longum subsp. longum CECT 7894 strain to encompass all main characteristics desirable for a probiotic composition to exert a beneficial effect in the human gut, especially when suffering from intestinal barrier dysfunction. These include resistance to gastrointestinal conditions (such resistance to gastric stress and bile salts), long-term stability, belonging to a species present among all stages of life and an outstanding capacity to produce polyP while proliferating. Therefore, probiotic formulas containing B. longum subsp. longum CECT 7894 according to the invention are useful for the improvement of any clinical condition where intestinal permeability is impaired.

Accordingly, the invention relates to a probiotic composition comprising Bifidobacterium longum subsp. longum strain deposited under the Budapest Treaty in the Spanish Type Culture Collection (CECT) under accession number CECT 7894, or a bacterial strain derived thereof, for use in a method of treating, preventing, or ameliorating an intestinal barrier dysfunction or associated condition, or symptoms, complications and/or sequela thereof in a subject in need thereof, by producing polyphosphate, wherein the derived bacterial strain:
(a) has a genome at least 99% identical to the genome of the correspondent deposited strain; and
(b) retains the ability of the correspondent deposited strain to produce polyphosphate.

“Bifidobacterium longum subsp. longum strain deposited under the Budapest Treaty in the Spanish Type Culture Collection (CECT) under accession number CECT 7894, or a bacterial strain derived thereof, wherein the derived bacterial strain: (a) has a genome at least 99% identical to the genome of the correspondent deposited strain; and (b) retains the ability of the correspondent deposited strain to produce polyphosphate” is hereinafter abbreviated as B. longum CECT 7894 or a bacterial strain derived thereof.

In another aspect, the invention provides a probiotic composition comprising B. longum CECT 7894 or a bacterial strain derived thereof, for use in the treatment of increased intestinal permeability and associated conditions in a subject, wherein the treatment of increased intestinal permeability is by producing polyphosphate, and wherein the associated conditions are non-intestinal conditions.

It is herein understood that the probiotic composition is useful in treating an intestinal barrier dysfunction, particularly increased intestinal permeability, and that it is also useful in treating an associated condition itself, i.e., a condition associated to the intestinal barrier dysfunction, particularly increased intestinal permeability. This can be alternatively expressed as a probiotic composition for use in treating the conditions described herein by treating increased intestinal permeability, by producing polyphosphates.

Another aspect of the invention relates to a combination comprising:
(i) B. longum CECT 7894 or a bacterial strain derived thereof, and
(ii) at least one human milk oligosaccharide,
   wherein the combination is configured for simultaneous, separate or sequential administration.

This aspect can alternatively be formulated as to a probiotic composition comprising B. longum CECT 7894 or a bacterial strain derived thereof as described herein, for use in combination with at least one human milk oligosaccharide, wherein the combination is configured for simultaneous, separate or sequential administration.

In another aspect, the invention relates to the combination as provided herein, for use in the treatment of increased intestinal permeability and associated conditions in a subject, wherein the treatment of increased intestinal permeability is by producing polyphosphate, and wherein the associated condition is selected from the group consisting of an immune disorder or disease, a metabolic or cardiovascular disorder or disease, a neurological or psychiatric disorder or disease and a gastrointestinal disorder or disease.

In another aspect, the invention provides a composition comprising:
(i) B. longum CECT 7894 or a bacterial strain derived thereof; and
(ii) at least one human milk oligosaccharide.

The probiotic composition, the combination, and the compositions according to the aspects of the invention can be used for different medical applications/uses which are described herein in detail. All the uses described herein can be alternatively formulated as the use of any of the compositions described herein for the manufacture of a pharmaceutical composition, a nutraceutical composition, a veterinary composition, or a food product/nutritional composition for the treatment, prevention or amelioration of an intestinal barrier dysfunction or associated condition or symptoms, complications and/or sequela thereof disclosed herein. This may be also alternatively formulated as methods of treating, preventing or ameliorating an intestinal barrier dysfunction or associated condition, or symptoms, complications and/or sequela described herein of a subject in need thereof comprising administering to the subject the herein described compositions according to the aspects of the invention.

Terms used in the claims and aspects of the invention are understood in its widely and common meaning in this description. Nevertheless, they are defined hereinafter in the detailed description of the invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### DESCRIPTION OF DRAWINGS

FIG. 1 shows the growth curves of the studied strains. PolyP was extracted and quantified at 6 and 16 h. OD means optical density (measured at 595 nm) and t (h) means time in hours.

FIG. 2 shows polyP biosynthesis (nmol) of studied strains after 6 and 16 h of growth.

FIG. 3 shows neighbor-joining tree showing the relationship across PPK proteins in the investigated bifidobacterial strains.

FIG. 4 shows stability of B. longum subsp. longum KABP-042 (CECT 7894) in final product over time. Live bacteria in Log cfus are represented over time in months (t (m)).

FIG. 5 shows growth of B. longum subsp. longum KABP-042 (CECT 7894) in presence of the HMO Lacto-N-Tetraose (LNT), glucose (Gluc) and in absence of carbon source (C—). OD means optical density (measured at 595 nm) and t (h) means time in hours.

FIG. 6 shows the apparent permeability coefficient (Papp) (left) and transepithelial electrical resistance (TEER) (right) of the Caco-2 barrier exposed to B. longum CECT 7894 supernatants with high (sb_MEI) and low (sb_LP) amounts of polyP. Cells were exposed to MEM, non-fermented MEI and LP media as controls.

FIG. 7 shows the relative expression of HSP27 protein in Caco-2 cells exposed to B. longum CECT 7894 supernatants with high (MEI) and low (LP) amounts of polyP. HSP27 quantity was normalized to β-actin quantity (left). Correlation (Pearson r=0.87, p=0.01) of relative expression of HSP27 and the amounts of polyP expressed as nmoles P in the supernatants (right).

FIG. 8 shows the relative expression (RE) of tight junction proteins Zonula ocludens-1 (ZO1), Junctional adhesion protein-1 (JAM1) and occluding in Caco-2 cells exposed to B. longum CECT 7894 supernatants with high (mei) and low (lp) amounts of polyP. Expression was normalized to 18S rRNA and GADPH genes expression.

FIG. 9 shows the polyP biosynthesis (nmol) of B. longum CECT 7894 cultures incubated under different conditions for 6 and 16 h: Control (C), Breast milk (BM), LNT, Polyamines (Polya).

FIG. 10 shows growth of B. longum subsp. longum KABP-042 (CECT 7894) in presence of the supernatant of B. bifidum Bb01 cultured with HMO 2′-Fucosyl-lactose (SN B. bifidum 2′-FL), glucose (Gluc) and in absence of carbon source (C—). OD means optical density (measured at 595 nm) and t (h) means time in hours.

### DETAILED DESCRIPTION OF THE INVENTION

### Definitions

Probiotic: As used herein, this term refers to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. The probiotic may be a variant or a mutant strain of bacterium. Probiotic bacteria may be naturally mutated or genetically engineered modified to retain, enhance or improve desired biological properties, e.g., survivability to provide probiotic properties or to retain, enhance or improve probiotic properties.

Derived from: The terms “derived from”, “derivative”, “variant”, “mutant” (e.g., “mutant strain”), or any grammatical variant thereof, as used herein, refer to a component that is isolated from or made using a specified molecule/substance (e.g., a strain of the present disclosure). For example, a bacterial strain that is derived from a first bacterial strain (e.g., a deposited strain) can be a strain that is identical or substantially similar to the first strain. In the case of bacterial strains, the derived strain can be obtained by, e.g., naturally occurring mutagenesis, artificially directed mutagenesis, artificially random mutagenesis or other genetic engineering techniques, and it retains, enhances or improves at least one ability of the deposited strain.

Excipient/Carrier: These terms are used interchangeably and refer to an inert substance added to a e.g., pharmaceutical composition, to further facilitate administration of a compound, e.g., a bacterial strain of the present disclosure. Examples include, but are not limited to, calcium bicarbonate, calcium phosphate, various sugars and types of starch, cellulose derivatives, gelatin, vegetable oils, polyethylene glycols, and surfactants, including, e.g., polysorbate. The terms “physiologically acceptable excipient/carrier” and “pharmaceutically acceptable excipient/carrier” which may be used interchangeably refer to a substance or a diluent that does not cause significant irritation to an organism and does not abrogate the biological activity and properties of the administered bacterial compound. An adjuvant is included under these terms.

Composition: As used herein, this term refers to the different compositions and combinations according to the aspects of the invention. Further, it refers to product forms such as a mixture of at least one compound useful within the invention with an excipient/carrier. For example, “pharmaceutical composition” refers to a preparation of the bacteria of the invention with other components such as a pharmaceutically acceptable carrier and/or excipient. The pharmaceutical composition facilitates the administration of the compound to a patient or subject.

Identity: As used herein, this term refers to the overall conservation of the monomeric sequence between polymeric molecules, e.g., between DNA molecules and/or RNA molecules. The term “identical” without any additional qualifiers, implies the sequences are 100% identical (100% sequence identity). Describing two sequences as, e.g., “70% identical,” is equivalent to describing them as having, e.g., “70% sequence identity.”

Calculation of the percent identity of two polymeric molecules, e.g., polynucleotide sequences, can be performed, e.g., by aligning the two sequences for optimal comparison purposes (e.g., gaps can be introduced in one or both of a first and a second polynucleotide sequences for optimal alignment). In certain aspects, the length of a sequence aligned for comparison purposes is at least about 30%, at least about 40%, at least about 50%, at least about 60%, at least about 70%, at least about 80%, at least about 90% or about 100% of the length of the reference sequence. The bases at corresponding base positions, in the case of polynucleotides, are then compared.

The percent identity between the two sequences is a function of the number of identical positions shared by the sequences, taking into account the number of gaps, and the length of each gap, which can be determined using a mathematical algorithm. Suitable software programs are available for alignment of both protein and nucleotide sequences. One suitable program to determine percent sequence identity is bl2seq, which performs a comparison between two sequences using either the BLASTN (used to compare nucleic acid sequences) or BLASTP (used to compare amino acid sequences) algorithm. Other suitable programs are, e.g., Needle, Stretcher, Water, or Matcher, part of the EMBOSS suite of bioinformatics programs. Sequence alignments can be conducted using methods known in the art such as MAFFT, Clustal (ClustalW, Clustal X or Clustal Omega), MUSCLE, MAUVE, MUMMER, RAST, etc.

In certain aspects, the percentage identity (% ID) of a first sequence to a second sequence is calculated as % ID=100×(Y/Z), where Y is the number of amino acid residues or nucleobases scored as identical matches in the alignment of the first and second sequences (e.g., as aligned by visual inspection or a particular sequence alignment program) and Z is the total number of residues in the second sequence. When comparing complete or near complete genomic nucleobase sequences, % ID is sometimes referred to as ANI (Average Nucleotide Identity). Calculating ANI usually involves the fragmentation of genome sequences, followed by nucleotide sequence search, alignment, and identity calculation.

Prevent: The terms “prevent,” “preventing,” “prophylaxis” and variants thereof as used herein, refer, e.g., to
(i) partially or completely delaying onset of a disease, disorder and/or condition disclosed herein;
(ii) partially or completely delaying onset of one or more symptoms, features, or clinical manifestations, complications, or sequelae of a particular disease, disorder, and/or condition disclosed herein;
(iii) partially or completely delaying onset of one or more symptoms, features, or manifestations, complications, or sequelae of a particular disease, disorder, and/or condition disclosed herein;
(iv) partially or completely delaying progression from a particular disease, disorder and/or condition disclosed herein; and/or
(v) decreasing the risk of developing pathology associated with the disease, disorder, and/or condition disclosed herein.

Subject: The terms “subject”, “patient”, “individual”, and “host”, and variants thereof are used interchangeably herein and refer to any mammalian subject, particularly humans, but also including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired. The compositions described herein are applicable to both human therapy and veterinary applications.

Infant: The term “infant” shall be understood in this description as the very young offspring of a human or animal, e.g., a child under the age of 1 year. When applied to humans, the term is considered synonymous with the term “baby”. The term “child” refers to a human between the stages of birth and puberty. “Young child” refers to a child aged between one and seven years and “toddler” between one and three years. However, in this description, the terms “infant”, “baby”, “young child” and “toddler” are considered synonymous and are used interchangeably.

Non-infant human or non-infant: These terms as used herein, refer to a human older than seven years. A non-infant human can be a teenager, an adult, or an elderly person (above 65 years of age). In this category, athletes and non-infant fragile people are also included.

Subject in need thereof: As used herein, “subject in need thereof” includes subjects, such as mammalian subjects, that would benefit from administration of the compositions of the disclosure.

Therapeutically effective amount: The terms “therapeutically effective dose” and “therapeutically effective amount” are used to refer to the amount of a composition of the present disclosure that is sufficient to a produce a desired therapeutic effect, pharmacologic and/or physiologic effect on a subject in need thereof. Particularly, the terms refer to an amount of a compound that results in prevention, delay of onset of symptoms, or amelioration of symptoms of a condition, e.g., diarrhea. A therapeutically effective amount can, e.g., be sufficient to treat, prevent, reduce the severity, delay the onset, and/or reduce the risk of occurrence of one or more symptoms of a disease or condition associated with a compromised gut barrier function. A therapeutically effective amount, as well as a therapeutically effective frequency of administration, can be determined by methods known in the art and discussed below.

Treatment: The terms “treat,” “treatment,” “therapy,” as used herein refer to, e.g., the reduction in severity of disease or condition disclosed herein; the mitigation/amelioration or elimination of one or more symptoms, complication, or sequelae associated with a disease disclosed herein (e.g., an intestinal barrier dysfunction or associated condition); the provision of beneficial effects to a subject with a condition/disease disclosed herein, without necessarily curing the disease or condition. The term also includes prophylaxis or prevention of a disease or condition or symptoms, complications, or sequelae thereof. Therefore, the expression “treating” as used herein, encompasses treating, preventing or ameliorating a disease, or symptoms, complications and/or sequela thereof.

The term refers to a clinical or nutritional intervention to prevent the disease or condition; cure the disease or condition; delay onset of the disease or condition; delay onset of a symptom, complication or sequela; reduce the seriousness of the disease or condition; reduce the seriousness of a symptom, complication, or sequela; improve one or more symptoms; improve one or more complications; improve one or more sequelae; prevent one or more symptoms; prevent one or more complications; prevent one or more sequelae; delay one or more symptoms; delay one or more symptoms; delay one or more complications; delay one or more sequelac; mitigate/ameliorate one or more symptoms; mitigate/ameliorate one or more complications; mitigate/ameliorate one or more sequelae; shorten the duration one or more symptoms; shorten the duration one or more complications; shorten the duration of one or more sequelae; reduce the frequency of one or more symptoms; reduce the frequency of one or more complications; reduce the frequency of one or more sequelae; reduce the severity of one or more symptoms; reduce the severity of one or more complications; reduce the severity of one or more sequelae; improve the quality of life; increase survival; prevent a recurrence of the disease or condition; delay a recurrence of the disease or condition; or any combination thereof, e.g., with respect to what is expected in the absence of the treatment with the composition of the present disclosure.

Dietary management and/or dietary secondary prevention: These terms refer to exclusive or partial feeding of patients who, because of a disease, disorder or medical condition they are suffering from: either have a limited, impaired or disturbed capacity to take, digest, absorb, metabolize or excrete ordinary food or certain nutrients contained therein, or metabolites, or have other medically determined nutrient requirements. In this description, “treating” or “treatment” encompasses dietary management and/or dictary secondary prevention.

Symptom: As used herein, this term refers to subjective or physical sign, indication, or evidence of disease or physical disturbance observed by the subject. In general, the term refers to any morbid phenomenon or departure from the normal in structure, function, or sensation, experienced by the patient and indicative of disease. Symptoms are felt or noticed by the individual experiencing the symptom, but may not easily be noticed by others. In some embodiments, a symptom can be a mild symptom, a moderate symptom, or severe symptom. As used herein, the term “mild symptom” refers to a symptom that is not life threatening and does not require, e.g., intensive care treatment. As used herein, the term “moderate symptom” refers to a symptom that requires monitoring because it may become life threatening and may require, e.g., hospitalization. As used herein, the term “severe symptom” refers to a symptom that is life threatening and requires, e.g., intensive care treatment.

Complication: As used herein, this term refers to a pathological process or event occurring during a disease or condition that is not an essential part of the disease or condition; where it may result from the disease/condition or from independent causes. For instance, treatment of medical conditions with antibiotics or non-steroidal anti-inflammatory drugs can result in epithelial injury in the intestine as a side effect, leading to increased permeability. This increased permeability can lead to an increased risk of allergic, inflammatory or metabolic diseases as long-term complications. In some aspects, a complication can be temporary. In some aspects, a complication can be chronic or permanent. As used herein, the term “sequela” refers to a long term, chronic, or permanent complication.

Intestinal barrier: As used herein, this term refers to a functional entity separating the intestinal lumen from the inner host, and consisting of mechanical elements (mucus, epithelial layer), humoral elements (defensins, IgA), immunological elements (lymphocytes, innate immune cells), muscular, neurological elements and microbiota.

Intestinal permeability: As used herein, this term refers to a functional feature of the intestinal barrier at given sites, measurable, inter alia, by analyzing flux rates across the intestinal wall as a whole or across wall components. Intestinal permeability refers to the control of material passing from inside the gastrointestinal tract through the cells lining the gut wall, into the rest of the body. A healthy intestine exhibits selective permeability, which allows nutrients to pass through the gut while also maintaining a barrier function to keep potentially harmful substances (such as antigens) from leaving the intestine and migrating to the body more widely.

Normal intestinal permeability: As used herein, this term refers to a stable permeability found in healthy individuals with no signs of intoxication, inflammation or impaired intestinal functions.

Intestinal barrier dysfunction: The terms “intestinal barrier dysfunction”, “impaired intestinal permeability”, “imbalanced intestinal permeability” and “abnormal intestinal permeability” are used interchangeably and refer to a disturbed permeability being non-transiently changed compared to the normal permeability leading to a loss of intestinal homeostasis, functional impairments and disease.

Increased intestinal permeability: As used herein, the term “increased intestinal permeability” refers to a condition where the junctions in the gut epithelial wall lose their integrity, allowing material from the lumen to translocate into the bloodstream, other organs, or the adipose tissue. When tight junctions of intestinal walls become loose, the gut becomes more permeable, which allow bacteria and toxins to pass from the gut into the bloodstream. This phenomenon is commonly referred to as e.g., “leaky gut”.

Increased intestinal permeability is a factor in several diseases, such as Crohn's disease, celiac disease, type 1 diabetes, type 2 diabetes, rheumatoid arthritis, spondyloarthropathies, inflammatory bowel disease, irritable bowel syndrome, schizophrenia, certain types of cancer, obesity, fatty liver, atopy and allergic diseases, among others. In the majority of cases, increased permeability develops prior to disease, but the cause-effect relationship between increased intestinal permeability in most of these diseases is not clear. For this reason, “intestinal barrier dysfunction (e.g., increased intestinal permeability and associated conditions” is used herein.

“Intestinal barrier”, “intestinal permeability”, “normal intestinal permeability”, “intestinal barrier dysfunction”, “impared/increased intestinal permeability” are terms also defined in Bischoff et al., 2014.

Human milk oligosaccharide: This term is abbreviated HMO and also known as “human milk glycan” and collectively refers to those oligosaccharides that are present in human milk, which constitute the third largest solid constituents in human milk, after lactose and fat. HMOs are short polymers of simple sugars that usually consists of lactose at the reducing end with a carbohydrate core that often contains a fucose or a sialic acid at the non-reducing end. HMOs are present in a concentration of 11.3-17.7 g/L in human milk, depending on lactation stages. Approximately 200 structurally different HMOs are known, and they can be categorized according to different classifications, e.g., into fucosylated, sialylated and neutral core HMOs. The composition of human milk oligosaccharides in breast milk is individual to each mother and varies over the period of lactation. The dominant oligosaccharide in 80% of all women is 2′-fucosyllactose, which is present in human breast milk at a concentration of approximately 2.5 g/L; other abundant oligosaccharides include lacto-N-tetraose, lacto-N-neotetraose, and lacto-N-fucopentaose.

Synthetic mixture: It means a mixture obtained by chemical and/or biological means, which can be chemically identical to the mixture naturally occurring, e.g., in mammalian milks. All compositions described herein are synthetic mixtures.

Nutritional composition: This term refers to a composition which nourishes a subject. This nutritional composition is usually to be taken orally or intravenously, and it usually includes a lipid or fat source and a protein source. Particularly the nutritional composition is a complete nutrition mix that fulfils all or most of the nutritional needs of a subject (e.g., an infant formula). Nutritional compositions comprise foodstuffs.

Infant formula: This term, as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The term “infant formula” encompasses the following forms without limitation:

Starter formula: It means a foodstuff intended for particular nutritional use by infants during the first sixth months of life.

Follow-up formula or follow-on formula: It may be given from the 6th month onwards. It constitutes the principal liquid element in the progressively diversified diet of this category of person.

Infant formula, follow on formula and starter infant formula can either be in the form of a liquid, ready-to-consumer or concentrated, or in the form of a dry powder that may be reconstituted to form a formula upon addition of water. Such formulae are well-known in the art.

Baby food: It means a foodstuff intended for particular nutritional use by infants or young children during the first years of life.

Infant cereal composition: It means a foodstuff intended for particular nutritional use by infants or young children during the first years of life.

Fortifier: It refers to liquid or solid nutritional compositions suitable for mixing with breast milk or infant formula.

Growing-up milk: It means a milk-based beverage adapted for the specific nutritional needs of young children.

Weaning period: It means the period during which the mother's milk is substituted by other food in the diet of an infant.

Enteral administration: It means any conventional form for delivery of a composition to a non-infant that causes the deposition of the composition in the gastrointestinal tract (including the stomach).

Oral administration: It means any conventional form for the delivery of a composition to a non-infant through the mouth. Accordingly, oral administration is a form of enteral administration.

### Probiotic Composition

In one embodiment, the probiotic composition comprises Bifidobacterium longum subsp. longum deposited under the accession number CECT 7894.

Strain Bifidobacterium longum subsp. longum CECT 7894 is described in WO2015018883A2, whose content is incorporated herein by reference in its entirety. The strain was deposited in the Spanish Type Culture Collection (CECT, Parc Científic de la Universitat de València, Carrer del Catedràtic Agustín Escardino Benlloch, 9, 46980 Paterna, Valencia, Spain) on Mar. 30, 2011 (30.03.2011) with accession number CECT 7894. Deposit was performed under the conditions of the Budapest Treaty, is viable and keeps all its features related to their deposit. It was deposited by the same applicant.

Bifidobacterium longum subsp. longum CECT 7894 (also referred in this description as KABP-042) was isolated from the faeces of a healthy breast-fed infant. In silico and in vitro analysis of CECT 7894 have been performed in order to study the probiotic attributes of this strain confirming the strain tolerates the challenges of human gastrointestinal tract (gastric conditions and bile salts) and adheres to intestinal epithelium. Genotypic analysis confirmed these features.

Human Milk Oligosaccharides (HMOs) are complex sugars found in human milk which utilization is strain-specific among Bifidobacteria. B. longum subsp. longum CECT 7894 has been found herein to be able to utilize in vitro the HMO Lacto-N-Tetraose (one of the most common HMOs found in breast milk). Concordantly, its genome harbors most of the typical HMO-degrading genes including acto-N-biosidase, beta-galactosidase, alpha-galactosidase, hexosaminidase and beta-glucuronidase. This analysis confirms the strain is adapted to HMOs utilization and thus to the infant gut.

Further, B. longum subsp. longum CECT 7894 has a versatile carbohydrate metabolism since other genes of its genome encode for Carbohydrate Active Enzymes (CAZy), suggesting its ability to degrade a wide range of complex substrates. In addition, genes encoding Lanthipeptide B, serpin and adhesins are also present in the genome of B. longum subsp. longum CECT 7894. Lanthipeptide B (Lantibiotic) is a class-I bacteriocin that exhibits strong antimicrobial activity against a range of gram-negative and gram-positive pathogenic bacteria. Serpins selectively inactivates human neutrophil and pancreatic elastases (proteases), resulting in an anti-inflammatory effect and contributing to maintaining gut homeostasis.

Overall, phenotypic and genotypic analysis of B. longum subsp. longum CECT 7894 herein confirms the strain is well adapted to the human gastrointestinal tract including the infant gut since it has the capacity to degrade HMOs.

As understood by the skilled person in the present context, a bacterial strain has been isolated from its natural environment, i.e., it is not present in its natural environment, so it is free from other organisms and substances present in the natural environment.

The emergence and spread of resistance to antimicrobials in bacteria pose a threat to human and animal health and present a major financial and societal cost. It has been found by whole genome sequence analysis that novel strain B. longum subsp. longum CECT 7894 does not possess transmissible antibiotic resistance genes to commonly used antibiotics. Overall, these results preclude the risk of a potential transfer of antibiotic resistance to pathogenic species.

It is clear that by using the deposited strain as starting material, the skilled person in the art can routinely, by conventional mutagenesis or re-isolation techniques, obtain further variants or mutants thereof that retain, enhance or improve the herein described relevant features and advantages of the strain forming the composition of the invention. Thus, the invention also relates to variants/mutants of the strain disclosed herein. In an embodiment, the probiotic composition comprises a bacterial strain derived from the strain Bifidobacterium longum subsp. longum CECT 7894, wherein the derived bacterial strain:
(a) has a genome with at least 99% average nucleotide identity (ANI) to the genome of the correspondent deposited strain CECT 7894; and
(b) retains, enhances or improves the ability of the correspondent deposited strain to produce polyphosphate.

In a particular embodiment, the bacterial strain derived from the deposited strain has a genome with at least 99% average nucleotide identity (ANI) to the genome of the correspondent deposited strain; more particularly, % of identity is 99.1%, 99.2%, 99.3%, 99.4%, 99.5%, 99.6%, 99.7%, 99.8% or 99.9%. Particularly the % of ANI is at least 99.5%. More particularly, % of ANI is 99.50%, 99.51%, 99.52%, 99.53%, 99.54%, 99.55%, 99.56%, 99.57%, 99.58%, 99.59%, 99.60%, 99.61%, 99.62%, 99.63%, 99.64%, 99.65%, 99.66%, 99.67%, 99.68%, 99.69%, 99.70%, 99.71%, 99.72%, 99.73%, 99.74%, 99.75%, 99.76%, 99.77%, 99.78%, 99.79%, 99.80%, 99.81%, 99.82%, 99.83%, 99.84%, 99.85%, 99.86%, 99.87%, 99.88%, 99.89%, 99.90%, 99.91%, 99.92%, 99.93%, 99.94%, 99.95%, 99.96%, 99.97%, 99.98% or 99.99%. In another embodiment, the % of ANI is at least 99.9%; particularly, % of ANI is 99.91%, 99.92%, 99.93%, 99.94%, 99.95%, 99.96%, 99.97%, 99.98% or 99.99%.

In some embodiments, the mutant is obtained by naturally occurring mutagenesis, artificially directed mutagenesis, or artificially random mutagenesis. In one particular embodiment, the bacterial strain derived from the deposited strain is obtained by using recombinant DNA technology. Thus, another aspect of the invention relates to a method to obtain a strain derived from the deposited strain, wherein the method comprises using the deposited strain as starting material and applying mutagenesis, and wherein the obtained variant or mutant further retains, enhances or improves at least one ability of the deposited strain disclosed herein.

The strain forming part of the composition of the invention may be in the form of viable cells. Alternatively, the strain may be in the form of non-viable cells. This could include thermally killed micro-organisms or micro-organisms killed by exposure to altered pH, sonication, radiation or high pressure. Product preparation is simpler with non-viable cells, as cells may be incorporated easily into dietary, pharmaceuticals or edible products, and storage requirements are much less limited than viable cells. A composition comprising the strain of the invention as non-viable cells can comprise products derived from the strain which are in the medium.

The strain disclosed herein is produced by cultivating (or fermenting) the bacteria in a suitable artificial medium and under suitable conditions. By the expression, “artificial medium” is understood to be a medium containing natural substances, and optionally synthetic chemicals such as the polymer polyvinyl alcohol which can reproduce some of the functions of serums. Common suitable artificial media are nutrient broths that contain the elements including a carbon source (e.g., glucose), a nitrogen source (e.g., amino acids and proteins), water and salts needed for bacterial growth. Growth media can be liquid form or often mixed with agar or another gelling agent to obtain a solid medium. The strain can be cultivated alone to form a pure culture, or as a mixed culture together with other microorganisms, or by cultivating bacteria of different types separately and then combining them in the desired proportions. After cultivation, and depending on the final formulation, the strain may be used as purified bacteria, or alternatively, the bacterial culture or the cell suspension may be used, either as such or after an appropriate post-treatment. In this description, the term “biomass” is understood to be the bacterial strain culture obtained after cultivation (or fermentation as a term synonymous to cultivation).

In a particular embodiment, the strain is fermented in an artificial medium and submitted to a post-treatment after fermentation, to obtain bacterial cells, and the resulting bacterial cells are in a liquid medium or in a solid form. Particularly, the post-treatment is selected from the group consisting of drying, freezing, freeze-drying, fluid bed-drying, spray-drying and refrigerating in liquid medium, and more particularly, is freeze-drying.

By the term “post-treatment” is to be understood in the present context, any processing carried out on the biomass with the aim of obtaining storable bacterial cells. The objective of the post-treatment is decreasing the metabolic activity of the cells in the biomass, and thus, slowing the rate of cellular deleterious reactions. As a result of the post-treatment, the bacterial cells can be in solid or liquid form. In solid form, the stored bacterial cells can be a powder or granules. In any case, both the solid and liquid forms containing the bacterial cells are not present in nature, hence, are not naturally-occurring, since they are the result of artificial post-treatment process(es). The post-treatment processes may in particular embodiments require the use of one or more of so-called post-treatment agents. In the context of the present invention, the expression “post-treatment agent” refers to a compound used to perform the herein described post-treatment processes. Among the post-treatment agents are to be included, without limitation, dehydrating agents, bacteriostatic agents, cryoprotective agents (cryoprotectants), inert fillers (also known as lyoprotectants), carrier material (also known as core material), etc., used either alone or in combination.

There are two basic approaches to decrease the metabolic activity of the bacterial cells, and thus, two approaches to carry out the post-treatment. The first one is decreasing the rate of all chemical reactions, which can be done by lowering the temperature by refrigerating or freezing using refrigerators, mechanical freezers, and liquid nitrogen freezers. Alternatively, decreasing the rate of all chemical reactions can be achieved by adding substances that inhibit the growth of the bacterial cells, namely a bacteriostatic agent, abbreviated Bstatic.

The second approach to carry out the post-treatment is to remove water from the biomass, a process which can involve sublimation of water using a lyophilizer. Suitable techniques to remove water from the biomass are drying, freeze-drying, spray-drying or fluid bed-drying. Post-treatments that result in solid form may be drying, freezing, freeze-drying, fluid bed-drying, or spray-drying.

The post-treatment is particularly freeze-drying, which involves the removal of water from frozen bacterial suspensions by sublimation under reduced pressure. This process consists of three steps: pre-freezing the product to form a frozen structure, primary drying to remove most water, and secondary drying to remove bound water. Due to objective and expected variability of industrial processes for manufacturing and isolation of lyophilized bacterial cultures, the latter commonly contain a certain amount of inert filler also known as lyoprotectant. Its role is to standardize the content of live probiotic bacteria in the product. The following inert fillers in commercially available lyophilized cultures are used: sucrose, saccharose, lactose, trehalose, glucose, maltose, maltodextrin, corn starch, inulin, and other pharmaceutically acceptable non-hygroscopic fillers. Optionally, other stabilizing or freeze-protecting agents like ascorbic acid, are also used to form a viscous paste, which is submitted to freeze-drying. In any case, the so-obtained material can be grinded to appropriate size, including to a powder.

Alternatively to having biomass preserved in solid form, biomass may be also preserved in liquid form. This may be done by adding a bacteriostatic agent as described above to stop bacteria growth to the culture medium or with an intermediate step of harvesting cells, re-suspending the pellet in saline solution with a bacteriostatic agent, and optionally refrigerating it.

Sometimes, as described for instance above in the fluid bed-drying process, the probiotic composition is subjected to an immobilization and/or coating, or encapsulation process in order to improve the shelf life and/or functionalities. Several techniques for immobilization, coating or encapsulation of bacteria are known in the art.

In other embodiments, the probiotic composition is formulated for sustained-release administration e.g., by means of the encapsulation in liposomes, microbubbles, microparticles or microcapsules and the like. The suitable sustained-release forms as well as materials and methods for their preparation are well known in the state of the art. Thus, the orally administrable form of any of the probiotic compositions of the invention is in a sustained-release form further comprising at least one coating or matrix. The sustained release coating or matrix includes, without limitation, natural semisynthetic or synthetic polymers, water-insoluble or modified, waxes, fats, fatty alcohols, fatty acids, natural, semisynthetic or synthetic plasticizers or a combination of two or more of the same. Enteric coatings can be applied using conventional processes known to those skilled in the art.

The effective amount of colony forming units (cfu) for the strain in the composition will be determined by the skilled in the art and will depend upon the final formulation. The term “colony forming unit” (“cfu”) is defined as the number of bacterial cells as revealed by microbiological counts on agar plates.

As known by the skilled person, the effective amount of colony units can also be measured by the effective amount of active fluorescent units. The term “active fluorescent unit” (“afu”) is defined as the number of bacterial cells as revealed by flow cytometry counts in a gate specific for fluorescence characteristics of presumed live cells. Therefore, the skilled person would consider the above-mentioned specific quantities of cfu to be about the same quantity of afu.

In one embodiment, the probiotic composition is a solid composition. In another embodiment, the probiotic composition is a liquid composition.

In another embodiment, the probiotic composition comprises: a freeze-dried bacterial biomass comprising from about 10⁵ cfu to about 10¹² cfu of the strain; more particularly from about 10⁸ cfu to about 10¹¹ cfu of the strain.

In an embodiment, the probiotic composition comprises a cryoprotectant. Particularly, the probiotic composition comprises at least one cryoprotectant that is an allergen-free cryoprotectant. In some embodiments, the probiotic composition comprises at least one cryoprotectant such as maltose, trehalose, mannitol (particularly, d-mannitol), saccharose, lactose, dextrose, sodium ascorbate, sodium citrate, L-cysteine, maltodextrin, anhydrous dextrose, starch, cellulose and inulin. In a particular embodiment, the cryoprotectant and/or the pharmaceutically acceptable carrier is selected from the group consisting of trehalose, D-mannitol, dextrose, sodium ascorbate, sodium citrate, L-cysteine, maltodextrin, starch, and cellulose. Particularly, the starch is corn, maize starch and/or potato starch.

More particularly, the composition further comprises a pharmaceutically acceptable carrier chosen from an emulsion, a suspension, a gel, a paste, granules, a powder, and a gum. Particularly, the carrier is an allergen-free carrier.

In some embodiments, the probiotic composition comprises one or more carriers selected from the group consisting of: maltodextrin, cellulose, starches of various types, inulin, lactose, or carrier with reduced water activity.

In a particular embodiment, the probiotic composition is a composition comprising:
a freeze-dried bacterial biomass comprising from about 10⁵ cfu to about 10¹² cfu of the strain;
a cryoprotectant and/or pharmaceutically acceptable carrier chosen from an emulsion, a suspension, a gel, a paste, granules, a powder, and a gum.

### Polyphosphate Production

In one embodiment, the production of polyphosphate of the strain Bifidobacterium longum subsp. longum CECT 7894 or a bacterial strain derived thereof is higher than the production of polyphosphate of a control strain, when the polyphosphate production is determined at 6 h and/or 16 h of culture by the following steps:
(a) culturing the strains inoculated at OD 0.1 in malic enzyme induction medium (MEI) containing (per liter, w/v): 0.5% yeast extract, 0.5% tryptone, 0.4% K₂HPO₄, 0.5% KH₂PO₄, 0.02% MgSO₄·7H₂O, 0.005% MnSO₄, 1 ml of Tween 80, 0.05% cysteine, and 0.5% glucose, at 37° C. and under anaerobic conditions;
(b) harvesting cells by centrifugation and lysis in 1 ml of 5% sodium hypochlorite with gentle agitation for 45 min at room temperature;
(c) centrifugating the insoluble material at 16,000 g for 5 min at 4° C. to obtain a pellet and washing twice with 1 ml of 1.5 M NaCl plus 1 mM EDTA at 16,000 g for 5 min at 4° C.;
(d) extracting polyP from the pellets with two consecutive washes with 1 ml of water and centrifugating at 16,000 g for 5 min at 4° C. between them;
(e) precipitating polyP in the pooled water extracts by adding 0.1 M NaCl and 1 volume of ethanol, followed by incubation on ice for 1 h;
(f) centrifugating at 16,000 g for 10 min and resuspending the polyP pellet in 50 μL of water;
(g) building a standard curve relating polyP-derived phosphate amount to fluorescence intensity, following the steps:
   i. hydrolyzing serial dilutions of a sample of polyP isolated from a polyP-producer control strain such as Lactobacillus plantarum WCFS1 (Alcántara et al. 2014) with a volume of 2 M HCl and incubation at 95° C. for 15 min;
   ii. neutralizing the dilutions by adding half volume of 2 M NaOH;
   iii. measuring the released phosphate with BIOMOL Green Kit to obtain the amount of phosphate in each dilution;
   iv. measuring the released phosphate by fluorescence using the 4′,6-diamidino-2-phenylindole, DAPI, at a final concentration of 10 μM in 50 mM Tris-HCl pH 7.5, 50 mM NaCl buffer with an excitation wavelength of 415 nm and emission at 550 nm in a fluorimeter to obtain the fluorescence value in each dilution; and
   v. building the standard curve with phosphate values obtained in (iii) and the corresponding fluorescence values obtained in (iv); and
(h) quantifying polyP from the resuspended fractions of step (f):
   1) measuring polyP by fluorescence using DAPI at a final concentration of 10 μM in 50 mM Tris-HCl pH 7.5, 50 mM NaCl buffer with an excitation wavelength of 415 nm and emission at 550 nm in a fluorimeter;
   2) calculating the amount of polyP by means of the standard curve; and
   3) expressing polyP value in nmol of phosphate.

Working EXAMPLE 1 (section 1.1.2 of Materials and Methods) herein provides a detailed description of an assay suitable to quantify polyP and consequently assess the capacity to produce polyP of a bacterial strain, as it is referred to steps (a)-(h) of the embodiment of the invention.

It is relevant to note that the descriptions and conditions of the polyP quantification assay disclosed in steps (a)-(h) of the embodiment of the invention are not limiting the scope of the invention. The assay is one suitable method to test the capacity of bacterial strains (e.g., B. longum subsp. longum CECT 7894) to produce polyP. The detailed conditions of this EXAMPLE 1 form herein a particular assay to determine if (derived) bacterial strains of interest comply with the criteria of the embodiment of the present invention.

Accordingly, based on the detailed assay described herein the skilled person is routinely able to repeat this assay to objectively determine whether specific bacterial strains of interest have the capacity to produce polyP of the embodiment of the present invention.

As said, the production of polyP can be quantified by means of the above-described method. Such method consists of three main steps, starting from polyP extraction from cells with sodium hypochlorite, dying of extracted polyP with DAPI and quantifying the fluorescence of the samples. PolyP amount is inferred from a standard curve which correlates the polyP-derived phosphate amounts with fluorescence units. This method is an indirect polyP quantification method through the measurement of phosphate by fluorescence.

In some embodiments, the quantification of polyP can be performed by means of alternative indirect polyP quantification methods. In a particular embodiment, the released phosphate from polyP hydrolyzation is measured with BIOMOL Green Kit for all samples to obtain the amount of phosphate, i.e., for both the control strain and the strain of the present invention. In another particular embodiment, the quantification of polyP is performed with the addition of PPK enzyme to obtain phosphate from polyP catabolism

In some embodiments, the production of polyP of the strain of the invention or a bacterial strain derived thereof is higher than the control strain when determined at 6 h and/or 16 h of culture, considering the same initial inoculum for all strains. Particularly, the production of polyP is higher when determined at 6 h and 16 h. In other embodiments, the production of polyP is higher when determined at one or more timepoints e.g., at 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h and/or 20 h of culture.

The control strain as it is understood herein and according to the invention is e.g., at least one of the following control strains: L. plantarum WCFS1 and L. paracasei JCM 1163 which are known to produce polyP; B. breve JCM 1273, B. adolescentis JCM 1275 and B. longum subsp. longum ATCC 15707 which are known to be able to remove phosphate; and B. scardovii DSMZ 13734 (BAA-773) that is known to harbor the gene ppk.

In a particular embodiment, the control strain is e.g., L. plantarum WCFS1, L. paracasei JCM 1163, B. breve JCM 1273, B. adolescentis JCM 1275, B. longum subsp. longum ATCC 15707 or B. scardovii DSMZ 13734 (BAA-773).

When the described assay is used, in some embodiments, the levels of polyP produced by B. longum subsp. longum CECT 7894 or a bacterial strain derived thereof at 6 h and 16 h are higher than the production of polyP of the control strain L. plantarum WCFS1 at the same point of time.

In some embodiments, the levels of polyP produced by B. longum subsp. longum CECT 7894 or a bacterial strain derived thereof at 6 h are at least e.g., 1.2-fold, 1.5-fold, 2-fold, 3-fold, 4-fold, 5-fold, 6-fold, 7-fold, 8-fold, 9-fold, 10-fold, 15-fold, 20-fold, 30-fold, 50-fold or 100-fold higher than the production of polyP of the control strain.

In a particular embodiment, the levels of polyP produced by B. longum subsp. longum CECT 7894 or a bacterial strain derived thereof at 6 h are at least 10-fold higher than the production of polyP of the control strain L. plantarum WCFS1. Particularly, the levels of polyP produced by B. longum subsp. longum CECT 7894 or a bacterial strain derived thereof at 6 h are at least 15-fold or 18-fold higher than the production of polyP of the control strain L. plantarum WCFS1.

In a particular embodiment, the levels of polyP produced by B. longum subsp. longum CECT 7894 or a bacterial strain derived thereof at 6 h are at least 3-fold higher than the production of polyP of the control strain B. breve JCM 1273.

In a particular embodiment, the levels of polyP produced by B. longum subsp. longum CECT 7894 or a bacterial strain derived thereof at 6 h are at least 4-fold higher than the production of polyP of the control strain B. adolescentis JCM 1275. Particularly, the levels of polyP produced by B. longum subsp. longum CECT 7894 or a bacterial strain derived thereof at 6 h are at least 4.5-fold higher than the production of polyP of the control strain B. adolescentis JCM 1275.

In a particular embodiment, the levels of polyP produced by B. longum subsp. longum CECT 7894 or a bacterial strain derived thereof at 6 h are at least 100-fold higher than the production of polyP of the control strain B. scardovii DSMZ 13734 (BAA-773). Particularly, the levels of polyP produced by B. longum subsp. longum CECT 7894 or a bacterial strain derived thereof at 6 h are at least 120-fold, 130-fold or 140-fold higher than the production of polyP of the control strain B. scardovii DSMZ 13734 (BAA-773).

In some embodiments, the levels of polyP produced by B. longum subsp. longum CECT 7894 or a bacterial strain derived thereof at 16 h are at least e.g., 1.2-fold, 1.5-fold, 2-fold, 2.5-fold, 3-fold, 4-fold, 5-fold, 6-fold, 7-fold, 8-fold, 9-fold, 10-fold, 15-fold, 20-fold, 30-fold, 50-fold or 100-fold, 200-fold, 300-fold, 400-fold, 500-fold or 600-fold higher than the production of polyP of the control strain.

In a particular embodiment, the levels of polyP produced by B. longum subsp. longum CECT 7894 or a bacterial strain derived thereof at 16 h is higher than the production of polyP of the control strain and the levels of polyP by the control strain at 16 h is non-existent. Particularly, the levels of polyP by the control strain at 16 h is non-existent when the control strain is L. plantarum WCFS1.

In a particular embodiment, the levels of polyP produced by B. longum subsp. longum CECT 7894 or a bacterial strain derived thereof at 16 h are at least 2-fold higher than the production of polyP of the control strain B. breve JCM 1273.

In a particular embodiment, the levels of polyP produced by B. longum subsp. longum CECT 7894 or a bacterial strain derived thereof at 16 h are at least 2.5-fold higher than the production of polyP of the control strain B. adolescentis JCM 1275.

In a particular embodiment, the levels of polyP produced by B. longum subsp. longum CECT 7894 or a bacterial strain derived thereof at 16 h are at least 500-fold higher than the production of polyP of the control strain B. scardovii DSMZ 13734 (BAA-773).

In a particular embodiment, the levels of polyP produced by B. longum subsp. longum CECT 7894 or a bacterial strain derived thereof at 6 h are at least 10-fold higher and at 16 h are higher than the production of polyP of the control strain L. plantarum WCFS1, wherein the production of polyP of the control strain L. plantarum WCFS1 and the levels of polyP by the control strain at 16 h is non-existent. Particularly, the levels of polyP produced by B. longum subsp. longum CECT 7894 or a bacterial strain derived thereof at 6 h are at least 15-fold or 18-fold higher and at 16 h are higher than the production of polyP of the control strain L. plantarum WCFS1, wherein the production of polyP of the control strain L. plantarum WCFS1 and the levels of polyP by the control strain at 16 h is non-existent.

In a particular embodiment, the levels of polyP produced by B. longum subsp. longum CECT 7894 or a bacterial strain derived thereof at 6 h are at least 100-fold higher and at 16 h are at least 500-fold higher than the production of polyP of the control strain B. scardovii DSMZ 13734 (BAA-773). Particularly, the levels of polyP produced by B. longum subsp. longum CECT 7894 or a bacterial strain derived thereof at 6 h are at least 120-fold, 130-fold or 140-fold higher and at 16 h are at least 500-fold higher than the production of polyP of the control strain B. scardovii DSMZ 13734 (BAA-773).

Combination of the Probiotic Composition with HMOs

The term probiotic composition as used herein, refers to a composition comprising Bifidobacterium longum subsp. longum strain CECT 7894 or a bacterial strain derived thereof in the terms described above.

As said, according to an aspect of the invention, the probiotic composition described herein can further comprise at least one human milk oligosaccharide in a combination.

Since the probiotic composition and HMOs can be formulated together in a single composition or in separate compositions, the embodiments described hereinafter refer to the “compositions of the present invention” or simply “compositions” to refer to the probiotic composition, the composition comprising HMOs and compositions including both.

In some embodiments the compositions of the invention comprise an additional probiotic different from B. longum CECT 7894 or a bacterial strain derived thereof, which is able to degrade HMOs, i.e., lacto-N-tetraose (LNT)). In a particular embodiment, the additional probiotic strain is a Bifidobacterium, more particularly, a Bifidobacterium bifidum or a Bifidobacterium longum subsp. infantis.

In a particular embodiment, the Bifidobacterium bifidum is B. bifidum deposited as CECT 30646. The strain was deposited in the Spanish Type Culture Collection (CECT, Parc Cientific de la Universitat de Valencia, Carrer del Catedràtic Agustín Escardino Benlloch, 9, 46980 Paterna, Valencia, Spain) on May 17, 2022 (17.05.2022) with accession number CECT 30646. Deposit was performed under the conditions of the Budapest Treaty, is viable and keeps all its features related to their deposit. It was deposited by the same applicant. B. bifidum CECT 30646 (also referred in this description as Bb01) was isolated from human breast milk.

In one embodiment, the compositions of the present invention comprise a HMO selected from the group consisting of a fucosylated oligosaccharide, a sialylated oligosaccharide, a N-acetyl-lactosamine and a combination thereof.

In a particular embodiment, the compositions comprise a fucosylated oligosaccharide (particularly 2′-fucosyllactose (2-FL) and/or difucosyllactose (DFL)) pand a N-acetyl-lactosamine (particularly, lacto-N-tetraose (LNT)).

HMOs can be isolated or enriched by well-known processes from milk(s) secreted by mammals including, but not limited to human, bovine, ovine, porcine, or caprine species and particularly, human. The HMOs can also be produced by well-known processes using microbial fermentation, enzymatic processes, chemical synthesis, or combinations of these technologies.

HMOs can be dissolved, emulsified, or suspended in e.g., water in the compositions of the invention.

In one embodiment, the HMOs are present in the compositions in a total amount of from 0.1 to 50 g/L or 0.3 to 5 g/L or 0.5 to 1 g/L, or 0.25 or 0.5 or 1 or 1.5 or 2 g/L.

### Fucosylated Oligosaccharide

The compositions according to the invention can comprise one or more fucosylated oligosaccharides. Particularly, the fucosylated oligosaccharides comprise 2′-fucosyllactose (2′-FL) and/or difucosyllactose (DFL).

In some embodiments, the fucosylated oligosaccharide is selected from the group comprising 2′-fucosyllactose (2′-FL), 3-fucosyllactose (3-FL), difucosyllactose (DFL), lacto-N-fucopentaose (i.e., LNFP I, II, III and V), lacto-N-difucohexaose (LNDFH I and II), lacto-N-difucohexaose III (LNDFH-III), fucosyl-lacto-N-hexaose (FLNH I and II), fucosyl-lacto-N-neohexaose (FLNnH), difucosyllacto-N-hexaose I, difucosyllacto-N-neohexaose (I and II) and fucosyl-para-lacto-N-hexaose (FpLNH I and II). Particular fucosylated oligosaccharides are 2-FL or DFL or a mixture thereof.

The fucosylated oligosaccharide can be isolated by chromatography or filtration technology from a natural source such as animal milks. Alternatively, it can be produced by biotechnological means using specific fucosyltransferases and/or fucosidase either through the use of enzyme-based fermentation technology (recombinant or natural enzymes) or microbial fermentation technology. In the latter case, microbes can either express their natural enzymes and substrates or can be engineered to produce respective substrates and enzymes. Single microbial cultures and/or mixed cultures can be used. Alternatively, fucosylated oligosaccharides are produced by chemical synthesis from lactose and free fucose. Fucosylated oligosaccharides are also available e.g., from Kyowa Flakko Kogyo of Japan.

Particularly, the compositions according to the invention comprise from 0.02 to 10 g of fucosylated oligosaccharide(s) per 100 g of composition on a dry weight basis, most particularly being 2FL, e.g., from 0.2 to 0.5 g or from 0.3 to 5 g of 2FL per 100 g of composition on a dry weight basis and particularly, 0.1 to 3 g of 2FL per 100 g of composition on a dry weight basis.

In some embodiments, the composition comprises an amount of 2FL in the following ranges or amount: 0.05 to 20 g/L or 0.1 to 5 g/L or 0.2 to 3 g/L or 0.1 to 2 g/L or 0.25 g/L to 1 g/L or 0.25 g/L or 1 g/L of composition, when the composition is in a ready-to-feed liquid form, or 0.05 to 20 g/L or 0.1 to 5 g/L or 0.2 to 3 g/L or 0.1 to 2 g/L or 0.25 g/L to 1 g/L or 0.25 g/L or 1 g/L (of the liquid diluted form) when the composition is in powder form and intended to be recomposed into a diluted liquid form, or the same as above multiplied by 2, 5, 10, 20, 50 or 100 when the composition is in the form of a concentrated composition intended to be diluted (respectively 2, 5, 10, 20, 50, or 100 times) into water or human breast milk or intended to be used directly as a concentrated form, or 0.04 g to 1.5 g/100 g of nutrition composition powder, or 0.08 to 1.2 g/100 g, or 0.1 to 1 g/100 g, or 0.2 to 0.8 g/100 g or 0.2 g/100 g or 0.4 g/100 g or 0.8 g/100 g or 1 g/100 g or 1 g/100 g of nutrition composition powder, when the nutritional composition is in the form of a dry powder.

### N-Acetyl-Lactosamine

In some embodiments the compositions of the invention comprise at least one N-acetyl-lactosamine, i.e., the compositions comprise N-acetyl-lactosamine and/or an oligosaccharide containing N-acetyl-lactosamine. Suitable oligosaccharides containing N-acetyl-lactosamine include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

In one embodiment the compositions according to the invention comprises a N-acetyl-lactosamine, particularly selected from the group comprising lacto-N-tetraose (LNT) and lacto-N-neotetraose (LNnT).

LNT and LNnT can be synthesized chemically by enzymatic transfer of saccharide units from donor moieties to acceptor moieties using glycosyltransferases. Alternatively, LNT and LNnT can be prepared by chemical conversion of keto-hexoses (e.g., fructose) either free or bound to an oligosaccharide (e.g., lactulose) into N-acetylhexosamine or an N-acetylhexosamine-containing oligosaccharide. N-acetyl-lactosamine produced in this way can then be transferred to lactose as the acceptor moiety. LNT can also be produced by microbial fermentation, e.g., with a genetically modified strain of E. coli K-12, as the one recently approved by EFSA.

Particularly, the compositions according to the invention comprise from 0.01 to 3 g of N-acetyl-lactosamine per 100 g of composition on a dry weight basis. Particularly it comprises 0.1 to 3 g of LNnT per 100 g of composition on a dry weight basis, e.g., from 0.1 to 0.25 g or from 0.15 to 0.5 g of LNnT per 100 g of composition on a dry weight basis.

In some embodiments, the compositions comprise an amount of LNnT in the following ranges or amount: 0.02 to 10 g/L or 0.05 to 2.5 g/L or 0.1 to 1.5 g/L or 0.05 to 1 g/L or 0.12 g/L to 0.5 g/L or 0.12 g/L or 0.5 g/L or 1 g/L of composition, when the composition is in a ready-to-feed liquid form, or 0.02 to 10 g/L or 0.05 to 2.5 g/L or 0.1 to 1.5 g/L or 0.05 to 1 g/L or 0.12 g/L to 0.5 g/L or 0.12 g/L or 0.5 g/L or 1 g/L (of the liquid diluted form) when the composition is in powder form and intended to be recomposed into a diluted liquid form, or the same as above multiplied by 2, 5, 10, 20, 50 or 100 when the composition is in the form of a concentrated composition intended to be diluted (respectively 2, 5, 10, 20, 50, or 100 times) into water or human breast milk or intended to be used directly as a concentrated form, or 0.02 g to 0.75 g/100 g of nutrition composition powder, or 0.04 to 0.6 g/100 g, or 0.0.5 to 0.5 g/100 g, or 0.1 to 0.4 g/100 g or 0.1 g/100 g or 0.2 g/100 g or 0.25 g/100 g or 0.5 g/100 g or 1 g/100 g or 3 g/100 g of nutrition composition powder, when the nutritional composition is in the form of a dry powder.

### Sialylated Oligosaccharides

The compositions according to the invention, in some embodiments, can comprise one or more sialylated oligosaccharides.

Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DS-LNT).

In one embodiment the composition according to the invention comprise a sialylated oligosaccharide, particularly selected from the group comprising 3′-sialyllactose and 6′-sialyllactose. More particularly, the compositions comprise both 3′-sialyllactose and 6′-sialyllactose, the ratio between 3′-sialyllactose and 6′-sialyllactose lying particularly in the range between 100:1 and 1:100, more particularly 10:1 and 1:10, even more particularly 5:1 and 1:2.

The 3′- and 6′-forms of sialyllactose can be isolated by chromatographic or filtration technology from a natural source such as animal milks. Alternatively, they can be produced by biotechnological means using specific sialyltransferases or sialidases, neuraminidases, either by an enzyme based fermentation technology (recombinant or natural enzymes), by chemical synthesis or by a microbial fermentation technology. In the latter case microbes can either express their natural enzymes and substrates or may be engineered to produce respective substrates and enzymes. Single microbial cultures or mixed cultures can be used. Alternatively, sialyllactoses can be produced by chemical synthesis from lactose and free N′-acetylneuraminic acid (sialic acid). Sialyllactoses are also commercially available for example from Kyowa Hakko Kogyo of Japan.

Particularly the composition according to the invention comprises from 0.05 to 10 g, more particularly 0.1 to 5 g, even more particularly 0.1 to 2 g of sialylated oligosaccharide(s) per 100 g of composition on a dry weight basis.

### Particular Product Forms

As understood by the skilled person in the present context, in relation to the herein provided combination, it is not essential that the two “compounds” as referred herein (i.e., B. longum CECT 7894 or a bacterial strain derived thereof, and HMOs) are administered e.g., simultaneously as a single intake or as a single composition or e.g., sequentially as two separate compositions. The important matter is that the two compounds can exert their effects together in the patient's body. Particularly, the two compounds are administered within a time frame e.g., the digestion period which can take up to eighteen hours in an adult.

Accordingly, the term “combination” relates herein to the various combinations of the two compounds e.g., in a single composition, in a combined mixture composed from separate compositions of the single compounds, such as a “tank-mix”, and in a combined use of the single compounds when applied in a sequential manner, i.e., one after the other with a reasonably short period, such as a few hours or in simultaneous administration. The order of administering B. longum CECT 7894 or a bacterial strain derived thereof, and the HMOs is not essential.

Thus, a combination of the probiotic composition and the HMOs can be formulated for its simultaneous, separate, or sequential administration. Particularly, if the administration is not simultaneous, the compounds are administered in a relatively close time proximity to each other. Furthermore, compounds are administered in the same or different dosage forms or by the same or different administration route, and particularly orally. In some embodiments, the combination of the two compounds are administered e.g.:
as a combination that is being part of the same composition, the two compounds being then administered always simultaneously;
as a combination of two units/compositions, each with one of the substances giving rise to the possibility of simultaneous, sequential or separate administration;
For instance, B. longum CECT 7894 or a bacterial strain derived thereof, is independently administered from the HMOs (i.e., in two units) but at the same time.

B. longum CECT 7894 or a bacterial strain derived thereof, and the HMOs can be formulated in any form as described in this description. Examples of different combinations are herein provided:

In an embodiment, the combination comprises a probiotic composition comprising B. longum CECT 7894 or a bacterial strain derived thereof, which is administered to breast-fed infants, the HMOs being present in the breast milk.

In another embodiment, the combination comprises a probiotic composition comprising B. longum CECT 7894 or a bacterial strain derived thereof, and an infant formula comprising HMOs. Thus, the composition comprising B. longum CECT 7894 or a bacterial strain derived thereof, is administered to formula-fed infants. Particularly, the composition comprising B. longum CECT 7894 or a bacterial strain derived thereof, is in form of oily drops.

In an embodiment, the combination comprises a single composition comprising B. longum CECT 7894 or a bacterial strain derived thereof, and the HMOs, in any of the product forms described in this description.

In an embodiment, the combination is for a non-infant and comprises a single composition comprising B. longum CECT 7894 or a bacterial strain derived thereof, and the HMOs. In another embodiment, the combination comprises a composition comprising the HMOs and a composition comprising the B. longum CECT 7894 or a bacterial strain derived thereof, e.g., in the form of an effervescent tablet or an energy bar.

As said, the combination can also include a further Bifidobacterium strain that can be formulated for the simultaneous, separate, or sequential administration with the other two compounds described herein.

### Uses of the Compositions

Embodiments of this section are referred to any “composition” according to the invention, namely, a probiotic composition comprising B. longum CECT 7894 or a bacterial strain derived thereof, and combinations and compositions including the probiotic composition and HMOs.

As discussed herein, the probiotic composition presents a high efficacy in producing polyphosphate while growing. The mechanisms of action of polyP are known to be linked to a protective effect over the epithelial cells by preventing intestinal permeability. Therefore, probiotic-derived polyP enhances intestinal barrier function and maintains intestinal homeostasis. The relation between the production of polyP and the protective effect in preventing/treating intestinal permeability has been demonstrated by the Examples provided herein (e.g., EXAMPLE 4). Further, it would be plausible for the skilled person that B. longum CECT 7894 through the production of polyP can have a positive effect in the intestinal barrier function and in the associated conditions described herein.

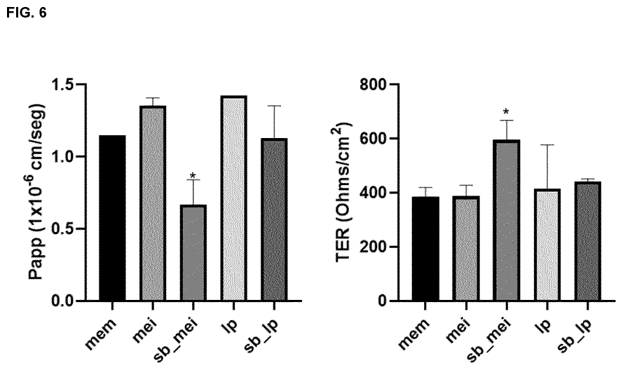

For example, Saiki et al., 2016 shows that the polyP extracted from the L. paracasei JCM 1163 suppresses the oxidant-induced intestinal permeability in mouse small intestine. Segawa et al., 2011 shows that polyPs inhibit mucosal permeability in an in vitro experiment with small intestine tissue. They first expose the tissue to an oxidizing agent that increases permeability and then add polyP to see the protective effect. Permeability is measured by quantifying the flow of mannitol. PolyPs reduce mannitol flow and therefore permeability. Similarly, Tanaka et al., 2015 demonstrates in vitro that polyPs reduce the flow of mannitol through Caco-2 intestinal epithelial cells, therefore, reducing permeability. Finally, Fujiya et al., 2020 studies permeability by measuring the resistance of the barrier with TEER (as evaluated herein, EXAMPLE 4, FIG. 6). They treat Caco-2 intestinal epithelial cells with TNF-alpha to increase permeability and then demonstrate that polyP improves resistance (improves TER).

Accordingly, the experimental data herein provided evidence that it is plausible that the probiotic composition would have significant positive effects in treating an intestinal barrier dysfunction and associated conditions in a subject in need thereof, by producing polyP.

In a particular embodiment, the probiotic composition is for use in a method of treating an intestinal barrier dysfunction. In an embodiment, the intestinal barrier dysfunction is associated to increased intestinal permeability. In an embodiment, the probiotic composition is for use in a method of treating increased intestinal permeability. In another embodiment, the probiotic composition is for use in a method of treating increased intestinal permeability and associated conditions.

In a particular embodiment, the subject is a mammal. In a more particular embodiment, the mammal is a human. Particularly, the human is an infant. In another embodiment, the human is a non-infant. In another embodiment, the human is selected from the group consisting of elderly people, pre-term infants, infants, athletes and fragile people.

In some embodiments, the intestinal barrier dysfunction (e.g., increased intestinal permeability) and associated conditions are related to pre-term birth, ageing, high-intensity physical activity, dietary imbalances, infection, drug treatment, or stress. In a particular embodiment, (e.g., increased intestinal permeability) and associated condition is related to ageing.

### Associated Conditions

A healthy gut barrier is considered to protect against bacteria translocation, bacteremia, autoimmunity, brain disorders, heart and liver diseases and obesity among other conditions. Intestinal barrier dysfunction has been strongly associated with immune disease, such as autoimmune diseases (Chron's disease, celiac disease, multiple sclerosis, rheumatoid arthritis, ulcerative colitis), other immune diseases (asthma, allergic rhinoconjunctivitis, atopic dermatitis, allergies/hypersensitivity such as food allergies/hypersensitivity), metabolic diseases such as non-alcoholic fatty liver disease, liver cirrhosis, diabetes type II and obesity, gastrointestinal diseases such as irritable bowel syndrome (IBS) or celiac disease, and a number of other diseases and conditions including pancreatitis, polycystic ovary syndrome and autism. Particularly, barrier disfunction due to mucosal injury is known to also arise from some drug treatments, such as oral antibiotics or non-steroidal anti-inflammatory drugs.

In some embodiments, the associated condition is an immune disorder or disease, a metabolic or cardiovascular disorder or disease, a neurological or psychiatric disorder or disease, or a gastrointestinal disorder or disease. Particularly, the immune disorder or disease is a non-intestinal immune disorder or disease. In another embodiment, the associated condition is a non-intestinal immune disorder or disease, a metabolic or cardiovascular disorder or disease, or a neurological or psychiatric disorder or disease.

In some embodiments, the intestinal barrier dysfunction (e.g., increased intestinal permeability) is associated to conditions occurring primarily in organs other than the intestine, referred herein as “non-intestinal conditions” or “conditions indirectly associated with the intestinal tract”. Of note, because of increased permeability, minimal overactivation or infiltration of immune cells can sometimes occur in some areas of the intestine in such conditions. However, such local events, if any, are asymptomatic and, to those skilled in the art, not the primary cause of health concern in patients with such conditions. Clear examples of such non-intestinal conditions to those skilled in the art are neurologic or psychiatric conditions (such as Alzheimer, autistic spectrum disorders, schizophrenia or depression), metabolic or cardiovascular conditions (such as prediabetes, diabetes, obesity, fatty liver disease, liver cirrhosis, atherosclerosis, hypertension, stroke or chronic heart failure) or immune disorders occurring at systemic level or in body locations distal from the intestine (such as lupus erythematosus, multiple sclerosis, immunosenescence, rheumatoid arthritis, asthma, allergic rhinoconjunctivitis, atopic dermatitis or other non-alimentary allergies/hypersensitivity). In such conditions, bacterial toxins (such as, but not limited to lipopolysaccharide (LPS) or trimethylamine N-oxide (TMAO)) can enter the systemic blood circulation thanks to increased permeability in the intestine, and cause inflammation and other deleterious health effects in organs located far away from the intestine, such as the heart, brain, lungs or skin, as well as the walls of the blood vessels or immune cells in various locations of the body.

The skilled in the art recognizes that increased intestinal permeability is associated to non-intestinal diseases described herein, such as: allergies, arthritis and metabolic diseases (Bischoff et al., 2014), psychiatric disorders (Kelly et al., 2015), hypertension and atherosclerosis (Verharr et al., 2020), cardiovascular disorders (Rogler et al., 2014), Alzheimer's disease (Jiang et al., 2017), obesity (Cox et al., 2015), atopic dermatitis (Pike et al., 1986), arthritis (Tajik et al., 2020) or metabolic diseases (Massier et al., 2021).

In a particular embodiment, the associated condition is an immune disorder or disease, particularly selected from the group consisting of autoimmune diseases, such as, but not limited to, Chron's disease, multiple sclerosis, rheumatoid arthritis, ulcerative colitis, and an allergic reaction/hypersensitivity (such as food allergy/hypersensitivity, asthma, atopic dermatitis or allergic rhinoconjunctivitis). Particularly, the immune disorder or disease is a non-intestinal immune disorder or disease, such as a non-intestinal autoimmune disease (particularly multiple sclerosis, lupus erythematosus or rheumatoid arthritis); immunosenescence, non-alimentary allergies/hypersensitivity, asthma, atopic dermatitis or allergic rhinoconjunctivitis.

In a particular embodiment, the associated condition is a metabolic or cardiovascular disorder or disease, which is particularly selected from the group including, but not limited to, stroke, chronic heart failure, atherosclerosis, hypertension, insulin resistance (prediabetes), diabetes, obesity, non-alcoholic fatty liver disease and liver cirrhosis.

In a particular embodiment, the associated condition is a neurologic or psychiatric disorder or disease, which is particularly selected from the group including, but not limited to, Alzheimer's disease, autistic spectrum disorders, schizophrenia and depression.

In some embodiments, the non-intestinal condition is selected from the group consisting of obesity, diabetes, insulin resistance, non-alcoholic fatty liver disease, liver cirrhosis, non-alimentary allergy/hypersensitivity, immunosenescence, multiple sclerosis, rheumatoid arthritis, lupus erythematosus, sarcopenia, asthma, allergic rhinoconjunctivitis, atopic dermatitis, Alzheimer's disease, atherosclerosis, hypertension, chronic heart failure, stroke, autistic spectrum disorders, schizophrenia and depression.s

In a particular embodiment, the associated condition is a gastrointestinal disorder or disease, which is particularly selected from the group including, but not limited to early inflammatory bowel disease (such as Crohn's disease, ulcerative colitis, pouchitis or lymphocytic colitis), irritable bowel syndrome (IBS), leaky gut syndrome, villous atrophy, necrotizing enterocolitis, intestinal ischemic injury, epithelial injury induced by non-steroidal anti-inflammatory drugs and celiac disease.

IBS, which is one of the most prevalent gastrointestinal disorders in high-income countries, is commonly associated to the presence of altered intestinal barrier. Alterations in the intestinal barrier have been reported to be associated with GI symptoms in IBS patients, such as diarrhea and abdominal pain. It seems that barrier dysfunction is an early event in IBS and may contribute to low-grade intestinal inflammation and increased visceral perception. Further, intestinal permeability in IBS subtypes such as diarrhea-predominant IBS (IBS-D) and post-infectious IBS are frequently related to altered intestinal barrier function.

Additionally, ulcerative colitis (UC) and Crohn's disease (CD), which are classified as chronic inflammatory bowel diseases (IBD), have similar symptoms and lead to digestive disorders including diarrhea, abdominal pain, rectal bleeding and weight loss. Epithelial integrity is disturbed in IBD patients that also display increased intestinal permeability. Intestinal barrier loss is a component that potentially contributes to a multi-hit mechanism of IBD pathogenesis. Moreover, many IBS patients with mucosal healing still have ongoing bowel symptoms, which have been associated with impaired intestinal permeability.

In another embodiment, the associated condition is characterized by microinflammation, vascular damage and/or dysbiosis of the gastrointestinal tract.

Particularly, the associated condition is directly associated with the intestinal tract. In a more particular embodiment, the intestinal barrier dysfunction or associated condition is selected from the group consisting of irritable bowel syndrome (IBS), inflammatory bowel disease (IBD), intestinal infection, gastric ulcer, diarrhea (e.g., gastric or infectious such as recurrent Clostridium difficile diarrhea), celiac disease, cancer associated with the digestive tract, colitis, ulcerative colitis, Crohn's disease, mitochondrial neurogastrointestinal encephalopathy (MNGIE), leaky gut syndrome, villous atrophy, necrotizing enterocolitis (NEC), intestinal ischemic injury, chronic enteropathy, chronic constipation, and intestinal mucosal injury. Particularly, intestinal barrier dysfunction due to mucosal injury is known to also arise from some drug treatments, such as oral antibiotics or non-steroidal anti-inflammatory drugs. Particularly, the associated condition is irritable bowel syndrome (IBS). Particularly, the associated condition is inflammatory bowel disease (IBD). Particularly, the associated condition is cancer. More particularly, the cancer in the digestive tract is selected from the group consisting of esophagus, stomach and colorectal cancer.

In some embodiments, the probiotic composition is for use in the treatment of at least one symptom, complication and/or sequela selected from the group consisting of abdominal pain, constipation, weight loss, rectal bleeding, sarcopenia, frailty, cachexia, gastrointestinal distress, cramping, bloating, flatulence, vomiting, nausea, gastric pain, fatigue, fever, altered absorption of specific nutrients, reduced appetite, systemic inflammation, and heat stroke. Particularly, the symptom, complication and/or sequela are selected from the group consisting of weight loss, sarcopenia, frailty, cachexia, fatigue, fever, systemic inflammation and heat stroke.

In an embodiment, the administration of the probiotic composition results in at least one outcome selected from the group consisting of:
reducing intestinal permeability, improving gastrointestinal barrier function, improving intestinal epithelium integrity or protecting intestinal mucosa;
reducing intestinal sensitivity or improving intestinal tolerability;
improving intestinal motility; and
maintaining intestinal balance.

The terms “reducing intestinal permeability”, “improving gastrointestinal barrier function”, “improving intestinal epithelium integrity” and “protecting intestinal mucosa” are understood as the adequate containment of undesirable luminal contents within the intestine.

The terms “reducing intestinal sensitivity” and “improving intestinal tolerability” are understood as a normal visceral response to pain stimuli.

The term “improving intestinal motility” is understood as regular movements of the gastrointestinal tract, and the transit of the contents within it.

The term “maintaining intestinal balance” is understood as an equilibrated intestinal ecosystem.

In another embodiment, the administration of the composition results in at least one outcome selected from the group consisting of:
lowering the level of intestinal permeability-related biomarkers;
alleviating or mitigating the increase of intestinal permeability-related biomarkers due to intestinal mucosal injury; and
decreasing tight junction protein levels increment in serum caused by intestinal mucosal injury.

Biomarkers may include circulating indicators such as intestinal fatty acid binding protein (I-FABP, also known as FABP-2), zonulin, claudin 3 (or other tight junction proteins), citrulline, lipopolysaccharide (LPS) or bacterial DNA; urine indicators such as oligosaccharides (e.g., lactulose, mannitol, sucralose, cellobiose, as well as ratios them, such as lactulose/mannitol ration), polyethylene glycols (PEGs), chromium-ethylenediaminetetraacetic acid (Cr-EDTA); or fecal markers including calprotectin, zonulin, alpha (a)-1-antitrypsin (AAT), diamine oxidase (DAO), or lipocalin-2 (LCN-2).

### Use in Infants

Increasing evidence implicates factors disrupting the early life microbiota with multiple disorders including inflammatory diseases such as allergies. Children with early exposure (first two years of life) to antibiotics have increased risk of allergic rhinitis, atopic dermatitis, childhood-onset asthma, celiac disease and obesity among other conditions. Further, babies born via C-section are more prone to allergic rhinoconjunctivitis and asthma than vaginal delivered babies, and reductions in Bifidobacteria are directly linked to atopic dermatitis and allergic asthma. Finally, infants fed with formula have a higher incidence of atopic dermatitis compared to breastfed infants. These results are consistent with the fact that gut microbiota plays a key role in shaping intestinal barrier structure and permeability and alterations in the gut microbiota are associated to increased intestinal permeability in several disorders.

Indeed, abnormal intestinal permeability is implicated in allergies. For instance, gut permeability is abnormally increased in 80% of children with food allergies and digestive manifestations. Further, impairment of the intestinal barrier is involved in the pathogenesis of atopic dermatitis. Likewise, babies with early allergic symptoms have increased gut permeability for proteins in comparison to non-allergic infants.

Thus, the probiotic composition described herein produces molecules (polyP) with the ability to restore the gut barrier, being a therapeutical option for treating allergies. Babies born by C-section, formula fed or administered with antibiotics and pre-term babies could also benefit from this probiotic treatment as prophylactic that may reduce allergy onset.

Accordingly, in an embodiment, the subject is an infant. Particularly, the infant is a pre-term infant, a fragile infant, an infant born with a subnormal birth weight, an infant subject of intrauterine growth retardation, an infant born by C-section, an infant administered with antibiotics, a formula-fed infant or a breast-fed infant. More particularly, the infant is a pre-term infant.

More particularly, the intestinal barrier dysfunction (e.g., increased intestinal permeability) and associated condition is related to pre-term birth, birth by C-section, formula fed, subnormal birth weight and/or antibiotics administration. In a particular embodiment, the intestinal barrier dysfunction (e.g., increased intestinal permeability) and associated condition is related to pre-term birth. In a particular embodiment, the intestinal barrier dysfunction (e.g., increased intestinal permeability) and associated condition is related to birth by C-section. In a particular embodiment, the intestinal barrier dysfunction (e.g., increased intestinal permeability) and associated condition is related to formula fed. In a particular embodiment, the intestinal barrier dysfunction (e.g., increased intestinal permeability) and associated condition is related to antibiotics administration.

Additionally, the probiotic composition of the invention is not only useful for the treatment of these conditions and the restoration of abnormal infant microbiota, but is also useful for the prevention of these conditions in the future by enhancing a healthy infant microbiota. Therefore, in a particular embodiment, the probiotic composition is for use in the prevention of conditions related to infants.

In some embodiments, the associated condition related to infants is selected from the group consisting of: Chron's disease, multiple sclerosis, lupus erythematosus, rheumatoid arthritis, ulcerative colitis, obesity, insulin resistance (prediabetes), diabetes, irritable bowel syndrome, celiac disease, early inflammatory bowel disease, an allergic reaction/hypersensitivity such as, but not limited to, food allergy/hypersensitivity, asthma, atopic dermatitis or allergic rhinoconjunctivitis, non-alcoholic fatty liver disease, autistic spectrum disorders, schizophrenia and depression.

In some embodiments, the associated condition related to infants is selected from the group consisting of: lupus erythematosus, multiple sclerosis, rheumatoid arthritis, non-alimentary allergy/hypersensitivity, asthma, atopic dermatitis, allergic rhinoconjunctivitis, insulin resistance (prediabetes), diabetes, obesity, non-alcoholic fatty liver disease, autistic spectrum disorders, schizophrenia and depression.

Particularly, the associated condition related to infants is selected from the group consisting of autistic spectrum disorders, non-alimentary allergy/hypersensitivity, asthma, atopic dermatitis, allergic rhinoconjunctivitis, insulin resistance (prediabetes), diabetes, fatty liver disease and obesity.

In a more particular embodiment, the associated condition is related to pre-term birth, and is allergy. In another embodiment, the associated condition is related to infants which are administered with antibiotics, and is selected from the group including, but not limited to, allergic rhinoconjunctivitis, atopic dermatitis, childhood-onset asthma, and obesity. In another embodiment, the associated condition is related to infants which are born by C-section, and is selected from the group consisting of allergic rhinoconjunctivitis, atopic dermatitis and asthma. In another embodiment, the associated condition is related to infants fed with formula, and is atopic dermatitis.

### Use in Athletes

Gastrointestinal distress symptoms, such as diarrhea, cramping, vomiting, nausea and gastric pain are common among athletes during high intensity training and competition. Stress of heat and oxidative damage during exercise causes disruption to intestinal epithelial cell tight junction proteins resulting in increased permeability to luminal endotoxins. Prolonged and strenuous physical exercise is related to an increase of the core temperature and intestinal permeability. Thus, the magnitude of exercise-induced hyperthermia is directly associated with the increase in intestinal permeability, which can trigger systemic inflammation that may affect physical performance and, in severe cases, induce heat stroke.

The administration of the probiotic composition described herein can counteract an exercise-induced leaky gut improving the integrity of the gut-barrier function and reducing gastrointestinal disturbances in athletes, which may improve their performance during exercise under high temperatures.

Accordingly, in a particular embodiment, the subject is an athlete. In a particular embodiment, the intestinal barrier dysfunction (e.g., increased intestinal permeability) and associated condition is related to high intensity physical activity.

In some embodiments, the probiotic composition of the invention is for use in a method of treating an intestinal barrier dysfunction (e.g., increased intestinal permeability) and associated condition, or symptoms, complications and/or sequela, selected from the group consisting of: diarrhea, cramping, vomiting, nausea, gastric pain, altered absorption of specific nutrients, systemic inflammation (that may affect physical performance) and, in severe cases, heat stroke.

### Use in Elderly

Ageing process is associated with a natural change in the gut microbiota composition, a low-grade chronic inflammation, and an increase in gut permeability, events which are all associated. Changes in gut microbiota comprise increased gut epithelial permeability, subsequent leakage of gut bacteria and their metabolites, and consequent inflammation. Further, local inflammation can be also directly modulated through changes in the microbiota.

Accordingly, in a particular embodiment, the subject is an elder or a fragile person. In a particular embodiment, the intestinal barrier dysfunction (e.g., increased intestinal permeability) and associated condition is related to ageing.

Particularly, the intestinal barrier dysfunction (e.g., increased intestinal permeability) and associated condition related to ageing is selected from the group consisting of constipation, diarrhea, sarcopenia, frailty, recurrent Clostridium difficile diarrhea, Alzheimer's disease, atherosclerosis, stroke, cancer and cachexia, and more particularly, sarcopenia, frailty, Alzheimer's disease, atherosclerosis, chronic heart failure, immunosenescence, and stroke.

### Product Forms Comprising the Compositions

Embodiments of this section are also referred to all compositions according to the invention, i.e., a probiotic composition comprising B. longum CECT 7894 or a bacterial strain derived thereof, a composition comprising HMOs and compositions including both.

### Pharmaceutical Forms

In some embodiments, the compositions described herein are in a pharmaceutical form, such as a capsule, a powder, a suspension, a tablet, a topical cream or an ointment.

The term “pharmaceutical form” is understood in its widest meaning, including any composition that comprises an active ingredient, in this case, the strain or the compositions described herein together with at least a pharmaceutically (also referred as nutraceutically or veterinary) acceptable excipient. The term “pharmaceutical form” is not limited to medicaments but includes e.g., pharmaceutical compositions, nutraceutical compositions or veterinary compositions. A pharmaceutical form can adopt different names depending on the product regulatory approval route and also depending on the country.

A nutraceutical composition can also be named e.g., as food supplement or dietary supplement. A nutraceutical composition is understood as a preparation or product intended to supplement the diet, made from compounds usually used in foodstuffs, which provide nutrients or beneficial ingredients that are not usually ingested in the normal diet or may not be consumed in sufficient quantities. Nutraceutical compositions are usually sold “over the counter”, i.e., without prescription.

In some embodiments, the compositions are formulated as pharmaceutical form in which the strain is the only active agent or is mixed with one or more other active agents and/or are mixed with pharmaceutically/nutraceutically/veterinary acceptable excipients. Particularly, the additional active agent or agents are other probiotic bacteria which are not antagonistic to the strain forming the composition of the invention. Depending on the formulation, the strain may be added as purified bacteria, as a bacterial culture, as part of a bacterial culture, as a bacterial culture which has been post-treated, and alone or together with suitable carriers or ingredients. Examples of other active ingredients to be added to the compositions are prebiotics such as fructo-oligosaccharides (e.g., inulin), galacto-oligosaccharides, xylo-oligosaccharides, arabinoxylan-oligosaccharides, pectins, beta-glucans, human milk oligosaccharides (e.g., Lacto-N-tetraose) or partially hydrolyzed guar gum.

The term “pharmaceutically/nutraceutical/veterinary acceptable” is art-recognized, and includes excipients, compounds, materials, compositions, carriers, vehicles and/or dosage forms which are, within the scope of sound medical judgment, suitable for use in contact with the tissues of a subject (e.g., human or animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical/nutraceutical/veterinary texts.

Thus, some embodiments of the invention relate to a pharmaceutical composition, a nutraceutical composition, and a veterinary composition comprising a composition described herein together with at least a pharmaceutically/nutraceutically/veterinary acceptable excipient as described above.

Some non-limiting examples of materials which may serve as pharmaceutically/nutraceutically/veterinary acceptable excipients or carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; or phosphate buffer solutions.

Excipients are selected, without limitation, from the group comprising: fillers/diluents/bulking agents, binders, antiadherents, disintegrants, coatings, anti-caking agents, antioxidants, lubricants, sweeteners, flavors, colors, or tensides.

Fillers are selected, without limitation, from the group comprising: inulin, oligofructose, pectin, modified pectins, microcrystalline cellulose, lactose, starch, maltodextrin, saccharose, glucose, fructose, mannitol, xylitol, non-crystallizing sorbitol, calcium carbonate, dicalcium phosphate, other inert inorganic and organic pharmacologically acceptable fillers, and mixtures of these substances. At dosage form of oral suspension, fillers or diluents are selected from the group comprising: vegetable oil, oleic acid, oleyl alcohol, liquid polyethylene glycol, other pharmacologically acceptable inert liquids, or mixtures of these substances.

Binders are used in solid dosage forms, e.g., to hold the ingredients in a tablet together, to ensure that tablets and granules can be formed with required mechanical strength, and to give volume to low active dose tablets. Binders in solid dosage forms like tablets are: lactose, sucrose, corn (maize) starch, modified starches, microcrystalline cellulose, modified cellulose (e.g., hydroxypropyl methylcellulose (HPMC) and hydroxyethylcellulose), other water-soluble cellulose ethers, polyvinylpyrrolidone (PVP) also known as povidone, poly-ethylene glycol, sorbitol, maltitol, xylitol and dibasic calcium phosphate; other suitable pharmacologically acceptable binders, or mixtures of these substances.

Antiadherents are used to reduce the adhesion between the powder (granules) and the punch faces and thus prevent sticking to tablet punches. They are also used to help protect tablets from sticking. The most commonly used is magnesium stearate.

As disintegrants and superdisintegrants in solid dosage forms like tablets and capsules, the following substances, without limitation, are used: cross-linked polyvinylpyrrolidone, sodium starch glycolate, sodium carboxymethyl cellulose, calcium carboxymethyl cellulose, and formaldehyde-casein, other suitable pharmacologically acceptable disintegrant and superdisintegrant, or their mixtures.

Coatings in the case of solid dosage forms, such as tablets and granules for capsule filling, protect the ingredients from deterioration by moisture in the air, make large, unpleasant-tasting tablets easier to swallow and/or in the case of enteric coatings ensure intact passage through a strong acidic medium of gastric juice (pH around 1), and which allow release in duodenum or ileum (small intestine). For most coated tablets, a cellulose ether hydroxypropyl methylcellulose (HPMC) film coating is used. Occasionally, other coating materials are used, e.g., synthetic polymers and co-polymers like polyvinylacetate phthalate (PVAP); co-polymers of methyl acrylate-metacrylic acid; co-polymers of methyl metacrylate-metacrylic acid; shellac, corn protein zein or other polysaccharides; waxes or wax-like substances such as beeswax, stearic acid; higher fatty alcohols like cetyl or stearyl alcohol; solid paraffin; glycerol monostearate; glycerol distearate, or their combinations. Capsules are coated with gelatin or hydroxypropyl methylcellulose.

Enteric coatings control the rate of drug release and determine where the drug will be released in the digestive tract. Materials used for enteric coatings include fatty acids, waxes, shellac, plastics, and plant fibers and their mixtures, also in combination with other above-mentioned coatings.

An anticaking agent is an additive placed in powdered or granulated materials to prevent the formation of lumps (caking) and for easing packaging, transport, and consumption. As anti-caking agents in solid dosage forms like tablets, capsules, or powders, the following are used: magnesium stearate, colloidal silicon dioxide, talc, other pharmacologically acceptable anticaking agents, or their mixtures.

Lubricants are used in solid dosage forms, in particular in tablets and capsules, to prevent ingredients from clumping together and from sticking to the tablet punches or capsule filling machine, and also in hard capsules. As lubricants talc or silica, and fats, e.g., vegetable stearin, magnesium stearate or stearic acid, and mixtures thereof, are the most frequently used lubricants in tablets or hard gelatin capsules.

Sweeteners are added to make the ingredients more palatable, especially in solid dosage forms, e.g., chewable tablets, as well as in liquids dosage forms, like cough syrup. Sweeteners may be selected from artificial, natural or synthetic or semi-synthetic sweeteners; non-limiting examples of sweeteners are aspartame, acesulfame potassium, cyclamate, sucralose, saccharine, sugars or any mixture thereof.

Flavors can be used to mask unpleasant tasting active ingredients in any dosage form. Flavorings may be natural (e.g., fruit extract) or artificial. For example, to improve: (1) a bitter product, mint, cherry or anise may be used; (2) a salty product, peach or apricot or liquorice may be used; (3) a sour product, raspberry; and (4) an excessively sweet product, vanilla.

Except auxiliary substances from the class of excipients, the formulation from the present invention can contain other pharmacologically active or nutritive substances including, but not limited, to vitamins, such as vitamin D (calciferol) in the pharmaceutically acceptable chemical form, salt or derivatives; minerals in the form of pharmaceutically and nutritive acceptable chemical form; and L-amino acids.

In each case the presentation of the composition will be adapted to the type of administration used by means known by the person skilled in the art. Thus, the composition may be presented in the form of solutions or any other form of clinically permissible administration and in a therapeutically effective amount. The composition can be thus formulated into solid, semisolid or liquid preparations, such as tablets, capsules, powders (such as those derived from lyophilization (freeze-drying) or air-drying), granules, solutions, suppositories, gels or microspheres. In a particular embodiment, the composition is formulated for administration in liquid form or in solid form.

In a particular embodiment, the composition is in solid form such as tablets, lozenges, sweets, chewable tablets, chewing gums, capsules, sachets, powders, granules, coated particles or coated tablets, tablet, pills, troches, gastro-resistant tablets and capsules, dispersible strips and films. More particularly, the composition is in form of a capsule, a powder, a tablet, a pill, lozenges, sachets, or granules. In an embodiment, the composition is in form of a powder which is put in contact with an aqueous phase to form a solution. The aqueous phase can comprise fibers such as inulin. The two components (the powder and the aqueous phase) can be in separate compartments/containers and the two components are mixed for in situ reconstitution.

In an embodiment, the composition is in form of gelatin capsules. In a particular embodiment, the composition is in the form of a vegetable capsule and comprises hydroxypropyl methylcellulose (HPMC).

In another embodiment, the composition is in liquid form such as oral solutions, drops, suspensions (e.g., oil), emulsions and syrups. Particularly, the composition is in form of drops. More particularly, the composition is in form of oily drops.

In some embodiments, the composition is in the form of an oily suspension to be administered alone or mixed with a liquid. The oily suspension comprises at least one edible oil such as olive oil, maize oil, soybean oil, linseed oil, sunflower oil or rice oil. The oil is present in a quantity of at least 70% weight/weight. In a particular embodiment, the oily suspension also comprises at least one excipient which is an emulsifier, stabilizer or anti-caking agent, in an amount of 0.1-15% w/w. Suitable agents are silicon dioxide, silica gel, colloidal silica, precipitated silica, talc, magnesium silicate, lecithin, pectin, starch, modified starches, konjac gum, xanthan gum, gellan gum, carrageenan, sodium alginate, mono- or diglycerides of fatty acids such as glycerol monostearate or glycerol monooleate and citric acid esters of mono- or diglycerides.

Particularly, the composition is in the form of an infant food supplement in the form of oily suspension, particularly in the form of oily drops. In a particular embodiment the oily suspension comprises sunflower oil and colloidal silica, particularly at 1% by weight, and the bacterial cells. In another embodiment the oily suspension comprises sunflower oil and an agent selected from lecithin, mono- or diglycerides of fatty acids, carrageenan and sodium alginate, and the bacterial cells.

Particularly, the e.g., capsule, sachet or stick, tablet or pill have a weight of about 150 mg to about 8000 mg. More particularly, the capsule has a weight of about 200 mg to about 600 mg. More particularly, the sachet or stick has a weight of about 1.5 g to about 6 g. More particularly, the tablet or pill have a weight of about 400 mg to about 1200 mg.

Particularly, the e.g., spray, oily drops (e.g., sunflower oily drops) have a volume of about 3 ml to about 50 ml. More particularly, the spray has a volume of about 5 ml to about 50 ml. More particularly, the oil drops have a volume of about 3 ml to about 30 ml.

Regarding the preparation of the formulations of the present invention, it is within the scope of ordinary person skilled in the art and will depend upon the final dosage formulation. For instance, and without limitation, when the final dosage form is an oral solid one, such as tablets, capsules, powder, granules, oral suspension, etc. the process for preparation of solid dosage forms of the formulation includes homogenization of: (1) the active ingredient(s), comprising post-treated probiotic bacteria of the invention in an effective amount; (2) with one or more excipients to form homogeneous mixture which is, e.g., according to requirements, subjected to lubrication with magnesium stearate or other lubricants yielding final dosage form of powder. Such homogeneous powder is filled into ordinary gelatin capsules or, alternatively, into gastro-resistant capsules. In the case of tablets, they are manufactured by direct compression or granulation. In the first case, a homogeneous mixture of active ingredients and suitable excipients such as anhydrous lactose, non-crystallizing sorbitol, and others is prepared. In the second case, tablets are processed of the mixture in granulated form. Granules are prepared by granulation process of active ingredients of the formulation with suitable fillers, binders, disintegrants, and small amount of purified water. Such prepared granules are sieved and dried until the water content of <1% w/w.

Regarding the process for preparation of liquid dosage forms (e.g., oral suspension), it involves homogenization of the active ingredient(s) of the formulation comprising post-treated probiotic bacteria of the invention in an effective amount in an inert liquid diluent (filler) such as various vegetable oils like sunflower, soybean or olive oil; oleic acid; oleyl alcohol; liquid polyethylene glycols like PEG 200, PEG 400 or PEG 600; or other inert pharmacologically acceptable liquids. The process further involves treatment of homogeneous mixture with one or more processes selected from the group comprising: (1) stabilization of the formulation, by addition and homogenization of suspension stabilizers like beeswax, colloidal silicon dioxide, etc.; (2) sweetening of the formulation, by addition and homogenization of sweetener; (3) flavoring of the formulation, by addition and homogenization of flavoring.

### Food Products/Nutritional Compositions

In some embodiments, the composition is in the form of a food product or an edible composition, such as infant formulas or food, milk-based fermented products (e.g., yogurt, cheese, curd), vegetable-based fermented products, breads, bars (e.g., energetic bars), spreads, biscuits, syrups, beverages, dressings, sauces, fillings, soups, ice creams, oils, dressings or confectionaries.

The term “food product or edible composition” are used herein in its broadest meaning, including any type of product, in any form of presentation, which can be ingested by an animal, particularly a human, but excluding pharmaceutical, nutraceutical and veterinary products.

Particularly, the composition is included in an infant formula or food. Particularly, the composition is included in a beverage.

Examples of other food products are meat products, chocolate spreads, fillings and frostings, chocolate, confectionery, baked goods, sauces and soups, fruit juices and coffee whiteners. The food product particularly comprises a carrier material such as oatmeal gruel, lactic acid fermented foods, resistant starch, dietary fibers, carbohydrates, proteins and glycosylated proteins. In a particular embodiment the strain of the invention is encapsulated or coated. Particularly, milks can be either of animal or vegetable origin.

In an embodiment, the food product or edible composition is a nutritional composition, commonly used in the field of infant nutrition but also used in elderly and fragile groups.

In a particular embodiment, the composition of the invention is an infant formula. In some embodiments, the compositions are e.g., a starter infant formula, a baby food, an infant cereal composition, a follow-on formula or a growing-up milk, or a fortifier. The composition can also be for use before and/or during a weaning period.

In one embodiment the nutritional composition can be a complete nutritional composition or a supplement for aging, elderly or fragile persons. In some embodiments, the composition of the invention is e.g., a rehydration solution or a dietary maintenance or supplement for elderly individuals, athletes or immunocompromised individuals.

The composition according to the invention can be completed composition provide 100% or a majority of the nutritional needs of the target populations (e.g., in term of caloric needs; or in terms of vitamin or minerals needs, in in term of protein, lipids or carbohydrate needs). Alternatively, the composition of the invention can be a supplement to be consumed in addition to a regular diet). In that case however the dosage and overall consumption of the composition is adapted to provide the claimed benefit on emotional processing (e.g., proportionally to the caloric load and to the subject caloric needs).

The use of a composition of the invention can encompass cases where the composition is a supplement, preferably provided in the form of unit doses (e.g., a tablet, a capsule, a sachet of powder, etc.). In one embodiment the composition is a supplement to human breast feeding. The unit dosage form can contain acceptable carriers, e.g., phosphate buffered saline solution, mixtures of ethanol in water, water and emulsions such as an oil/water or water/oil emulsion, as well as various wetting agents or excipients. Examples of carriers and excipients are described above in this description.

The composition can be in the form of a powder composition e.g., intended to be diluted with water or mixed with milk (e.g., human breast milk), or ingested as a powder. In one embodiment the composition of the invention is in liquid form; either ready-to-drink or to be diluted in water or mixed with milk (e.g., human breast milk).

The composition can be in the form of a ready-to-feed liquid or may be a liquid concentrate or powdered formula that can be reconstituted into a ready-to-feed liquid by adding an amount of water that results.

### Administration

In some embodiments, the composition is administered in a single dose or repeated dose at specific time intervals, e.g., can be administered daily for a specific number of days or according to a specific dosing schedule. Particularly, the composition is administered during from 10 days to 90 days. More particularly, it is administered during from 10 days to 60 days or from 15 to 45 days, more particularly during 30 days.

In some embodiments, the composition is administered from once every three days to thrice a day, particularly, once a day.

In some embodiments, the composition can be administered orally, rectally, parenterally, topically, ocularly, aurally, nasally, intravaginally or to the buccal cavity, to give a local and/or a systemic effect. Particularly, the composition is administered orally. In an embodiment, a unit dose of the compositions of the invention is administered orally, in any form described above, such as a tablet, capsule, or pellet, or as a powder or granules or as a gel, paste, solution, suspension, emulsion, syrup, bolus, electuary, or slurry, in an aqueous or nonaqueous liquid.

In one embodiment, the compositions are administered enterally. Methods of enteral administration include feeding through a naso gastric tube or jejunum tube, oral, sublingual and rectal. Thus, a unit dosage form of the compositions can also be administered by rectal suppository, aerosol tube, naso-gastric tube or direct infusion into the gastrointestinal tract or stomach, in elderly or fragile people.

In other embodiments, the composition can be administered by nasal inhalation, oral spray via or nasogastric route. In other embodiments, the composition can be administered in form of oral drops.

### EXAMPLES

### Example 1: Polyphosphate Biosynthesis Capacity of Bifidobacterium longum Subsp. Longum KABP-042 (CECT 7894)

### 1.1 Materials and Methods

### 1.1.1 Strains and Culture Conditions

The capacity to biosynthesize polyP was assessed in 19 strains (TABLE 1). Strains included B. longum subsp. longum KABP-042 (CECT 7894), other Bifidobacteria strains and other strains belonging to Lactobacillus group and Saccharomyces genus. Strains included infant and adult Human Residential Bacteria (HRB) strains and non-HRB strains from AB-Biotics S.L. collection or commercially available products.

Analysis included the following control strains. L. plantarum WCFS1 (Alcántara et al. 2014) and L. paracasei JCM 1163 (Saiki et al. 2016) which are known to produce polyP; B. breve JCM 1273, B. adolescentis JCM 1275 and B. longum subsp. longum ATCC 15707 which are known to be able to remove phosphate (Anand et al. 2019); and B. scardovii DSMZ 13734 (BAA-773) which is known to harbor the gene ppk (Qian et al. 2011).

Strains were isolated from commercial products when indicated by inoculating on appropriate agar plates. After culture, single colonies were grown for storage in glycerol stocks and species identity (ID) were confirmed by PCR amplification and Sanger sequencing of 16S rRNA gene. Control strains were purchased from the culture collections and species identity confirmed.

Bifidobacterial strains were pre-cultured in Man, Rogosa and Sharpe agar (MRS) with 0.05% cysteine (MRScys), at 37° C. and under anaerobic conditions. Lactobacilli strains were pre-cultured in MRS at 30° C. and under aerobic conditions. Saccharomyces boulardii CNCM I-754 was pre-cultured in YPD media at 37° C. under aerobic conditions with shaking.

For polyP production assay, malic enzyme induction (MEI) medium containing (per liter, w/v) 0.5% yeast extract, 0.5% tryptone, 0.4% K₂HPO₄, 0.5% KH₂PO₄, 0.02% MgSO₄·7H₂O, 0.005% MnSO₄, 1 ml of Tween 80, 0.05% cysteine, and 0.5% glucose was used (Alcántara et al. 2014). The strains unable to grow in MEI were grown in MRScys. Cultures were inoculated at OD (595 nm) 0.1 and each strain was grown under the conditions indicated above. Growth was monitored by measuring OD for 16 h.

TABLE 1
Characterization of strains. HRB, Human Residential Bifidobacteria; nHRB, non-
HRB; CECT, Spanish Type Culture Collection; DSMZ, German Collection of Microorganisms
and Cell Cultures; ATCC, American Type Culture Collection. Strains classified
as Control have some published evidence of polyP metabolism.

| Strain | Origin | HRB/nHBR | Classification | Source |
|---|---|---|---|---|
| Bifidobacterium longum subsp. | Breast-fed | Infant & adult | Probiotic | AB-Biotics |
| longum KABP-042 (CECT | infant gut | HRB | | collection |
| 7894) | | | | |
| B. longum subsp. longum | Adult gut | Infant & adult | Probiotic | Commercial |
| 36524 ™ | | HRB | | product |
| B. longum subsp. longum | Adult gut | Infant & adult | Control | CECT |
| ATCC 15707 | | HRB | | |
| B. longum subsp. longum | Breast-fed | Infant & adult | Probiotic | Commercial |
| BB536 | infant gut | HRB | | product |
| B. longum subsp. longum | Breast-fed | Infant & adult | Probiotic | AB-Biotics |
| ABP123 | infant gut | HRB | | collection |
| B. bifidum ABP671 | Breast-fed | Infant HRB | Probiotic | AB-Biotics |
| | infant gut | | | collection |
| B. breve ABP734 | Breast milk | Infant HRB | Probiotic | AB-Biotics |
| | | | | collection |
| B. breve M16-V | Infant gut | Infant HRB | Probiotic | Commercial |
| | | | | product |
| B. breve JCM 1273 | Infant gut | Infant HRB | Control | DSMZ |
| B. adolescentis JCM 1275 | Adult gut | Adult HRB | Control | DSMZ |
| B. animalis BB-12 | Dairy | nHRB | Probiotic | Commercial |
| | culture | | | product |
| B. scardovii BAA-773 | Human | nHRB | Pathogen | DSMZ |
| | blood | | (control) | |
| Lacticaseibacillus paracasei | Beer | NA | Control | DSMZ |
| JCM 1163 | | | | |
| Lactiplantibacillus | Human | NA | Control | ATCC |
| WCFS1 | saliva | | | |
| Lactiplantibacillus plantarum | Sour dough | NA | Probiotic | Commercial |
| 299v | | | | product |
| Levilactobacillus brevis KABP- | Human | NA | Probiotic | AB-Biotics |
| 052 | saliva | | | collection |
| (CECT 7480) | | | | |
| Lacticaseibacillus rhamnosus | Adult gut | NA | Probiotic | ATCC |
| GG | | | | |
| Limosilactobacillus reuteri | Derived | NA | Probiotic | Commercial |
| DSM 17938 | from | | | product |
| | ATCC | | | |
| | 55730 | | | |
| Saccharomyces boulardii | Tropical | NA | Probiotic | Commercial |
| CNCM I-754 | fruit | | | product |

1.1.2 Polyphosphate (polyP) Quantification

PolyP was isolated from cells by its resistance to hydrolysis with sodium hypochlorite as previously described (Alcántara et al. 2014). Cells were harvested by centrifugation and lysed in 1 ml of 5% sodium hypochlorite with gentle agitation for 45 min at room temperature. Insoluble material was pelleted by centrifugation at 16,000 g for 5 min at 4° C. and washed twice with 1 ml of 1.5 M NaCl plus 1 mM EDTA at 16,000 g for 5 min at 4° C. PolyP was extracted from the pellets with two consecutive washes with 1 ml of water and a centrifugation step at 16,000 g for 5 min at 4° C. between them. PolyP in the pooled water extracts was precipitated by adding 0.1 M NaCl and 1 volume of ethanol, followed by incubation on ice for 1 h. After centrifugation at 16,000 g for 10 min, the polyP pellet was resuspended in 50 μL of water.

A standard curve relating phosphate amount to fluorescence intensity was built to quantify the extracted polyP from the strains. As a first step, serial dilutions of a sample of polyP isolated from the polyphosphate-producer control strain Lactiplantibacillus plantarum strain WCFS1 (Alcántara et al. 2014) were prepared. Second, the dilutions were hydrolyzed with a volume of 2 M HCl, incubated at 95° C. for 15 min to release phosphate and then neutralized by adding half volume of 2 M NaOH. Third, the released phosphate from each dilution was quantified with BIOMOL Green Kit (Enzo Life Sciences) as recommended by the manufacturer. In parallel, the released phosphate from each dilution was dyed using 4′,6-diamidino-2-phenylindole (DAPI) at a final concentration of 10 μM in 50 mM Tris-HCl pH 7.5, 50 mM NaCl buffer and fluorescence was measured with an excitation wavelength of 415 nm and emission at 550 nm in a fluorimeter. Finally, a standard curve was built with phosphate values and the corresponding fluorescence values obtained.

Once the standard curve is obtained, polyP amounts from the samples can be quantified according to fluorescence values, without the need of using BIOMOL Green kit. Therefore, the quantification of polyP from the strain samples was indirectly measured by DAPI fluorescence using the standard curve. Firstly, the extracted polyP was measured by fluorescence using DAPI at a final concentration of 10 μM in 50 mM Tris-HCl pH 7.5, 50 mM NaCl buffer with an excitation wavelength of 415 nm and emission at 550 nm in a fluorimeter. Then, the amount of polyP was calculated as nmol of phosphate by means of the standard curve. At least three biological replicates were performed.

### 1.1.3 Detection of Ppk Gene by in Silico Analysis

Nucleotide sequences for ppk genes in Bifidobacteria and Lactobacilli species were retrieved from the NCBI with the accession numbers AE014295.3 (version 3, update date 31.01.2014, genome of B. longum NCC2705) and AL935263.2 (version 2, update date 28.02.2015, genome of L. plantarum WCFS1), respectively, and subjected to BLAST analysis against the genomes of study. Amino acid sequences of detected PPK proteins in Bifidobacterium species were aligned and a tree was constructed using ClustalW.

### 1.2. Results

The ability of B. longum subsp. longum KABP-042 (CECT 7894) to produce polyP and its associated growth was compared to 12 bifidobacterial strains belonging to 6 different species, 6 lactobacilli strains belonging to 5 species and 1 yeast strain (TABLE 1).

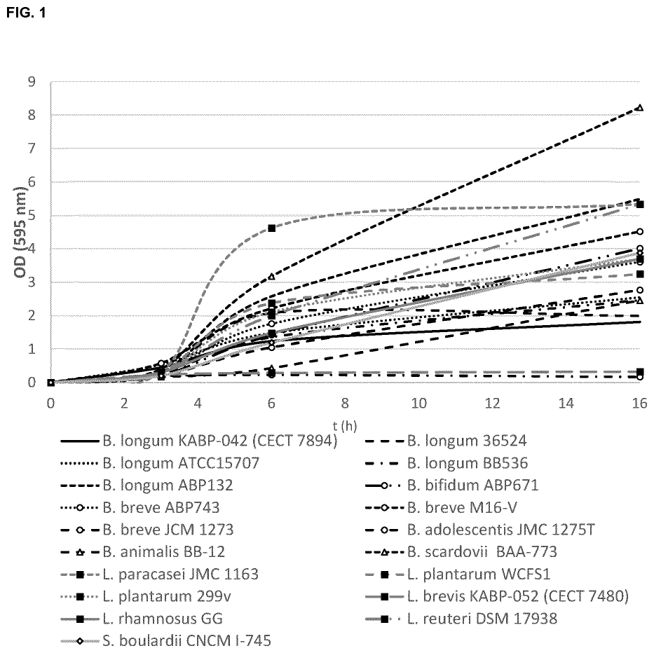

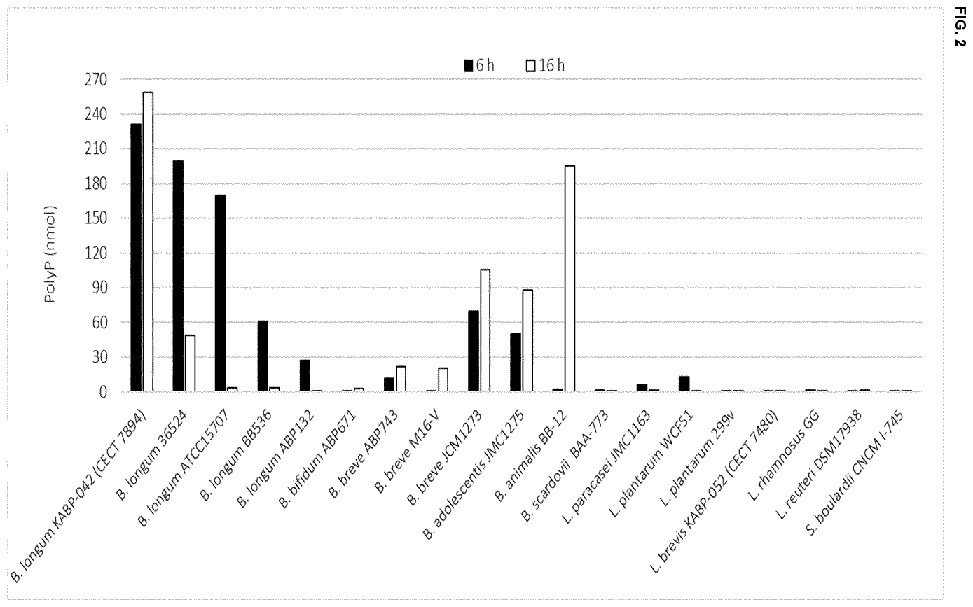

Strains were inoculated at the same OD (0.1) in MEI or MRScys and grown for 16 h. OD was monitored, and polyP formation was studied at 6 h and 16 h when significant growth was observed in most of the strains (FIG. 1). PolyP synthesis and OD values varied strongly between strains (FIG. 1, FIG. 2 and TABLE 2).

In general, Bifidobacteria showed a greater capacity to form polyP than lactobacilli strains. PolyP levels produced by L. plantarum 299v, L. brevis KABP-052 (CECT 7840), L. rhamnosus GG, L. reuteri DSM 17938 and S. boulardii CNCM I-754 cells were very low (<2 nmol at 16 h).

Among Bifidobacteria, all the strains were able to produce some amounts of polyP. However, B. bifidum ABP671, B. breve ABP734, B. breve M16-V and B. scardovii BAA-773 produced the lowest amounts (<25 nmol at 16 h, FIG. 2 and TABLE 2). This result indicated that polyP synthesis in Bifidobacteria was highly variable among different strains as observed previously in Lactobacilli.

Comparing polyP production at time 6 and 16 h, B. scardovii and all B. longum strains but B. longum subsp. longum KABP-042 (CECT 7894) showed greater values of polyP at 6 h than 16 h (FIG. 2 and TABLE 2). The rest of strains produced more polyP at 16 h while B. longum subsp. longum KABP-042 (CECT 7894) produced similar amounts at both time-points. Therefore, it can be concluded that polyP production in Bifidobacteria varies along the growth curve and this growth-related variation also depends on strain, highlighting the importance to analyze more than one time-point along the growth curve.

Notably, B. longum subsp. longum KABP-042 (CECT 7894) showed the greater capacity to produce polyP at 6 h (TABLE 2). Surprisingly, at 16 h B. longum subsp. longum KABP-042 (CECT 7894) also showed the best ability to form polyP. Interestingly, B. longum subsp. longum KABP-042 was the only B. longum strain showing this behavior, i.e., a high production of polyP was observed regardless of the age of the culture. Conversely, other polyP-producing strains showed the capacity only when the culture was young (e.g., B. longum subsp. longum ATCC 15707) or when it was old (e.g., B. animalis BB12). Thus, the capacity to produce polyP in a constant manner represents an additional advantage of the strain B. longum subsp. longum KABP-042 (CECT 7894).

It is noteworthy to mention that, unlike B. adolescentis JCM 1275, B. longum subsp. longum KABP-042 (CECT 7894) was able to proliferate while producing polyP. In addition, B. longum subsp. longum KABP-042 (CECT 7894) produced more polyP than other strains that were able to grow even more. This evinces B. longum subsp. longum KABP-042 (CECT 7894) has the highest potential to proliferate and colonize the gut while externing beneficial effects by the efficient production of the postbiotic molecule polyP.

Furthermore, B. longum subsp. longum KABP-042 (CECT 7894) was able to produce 140 times more polyP at 6 h than B. scardovii BAA-773 (1.6 vs 230.9 nmol, TABLE 2) which is known to express ppk (Qian et al., 2011). B. longum subsp. longum KABP-042 (CECT 7894) was able to produce 18 times more polyP than L. plantarum WCFS1 (12.7 vs 230.9 nmol, TABLE 2), which is known to produce polyP (Alcántara et al., 2018).

TABLE 2
PolyP quantification (nmol) and growth (OD₅₅₀) of
strains analyzed in the study at 6 h and 16 h.

| | 6 h | | 16 h | | Genome | |
|---|---|---|---|---|---|---|
| Strain | PolyP | OD | PolyP | OD | available | ppk |
| B. longum subsp. longum KABP- | 230.9 | 1.2 | 258.3 | 1.8 | Yes | + |
| 042 (CECT 7894) | | | | | | |
| B. longum subsp. longum | 199.0 | 2.1 | 49.0 | 2.0 | Yes | + |
| 36524 ™ | | | | | | |
| B. longum subsp. longum ATCC | 169.7 | 1.5 | 3.9 | 2.5 | Yes | + |
| 15707 | | | | | | |
| B. longum subsp. longum BB536 | 61.3 | 1.4 | 3.3 | 2.5 | No | NA |
| B. longum subsp. longum ABP123 | 27.0 | 2.6 | 0.4 | 5.5 | No | NA |
| B. bifidum ABP671 | 0.3 | 1.4 | 2.6 | 4.0 | No | NA |
| B. breve ABP734 | 11.9 | 1.8 | 22.0 | 3.6 | Yes | + |
| B. breve M16-V | 0.1 | 2.2 | 20.4 | 4.5 | No | NA |
| B. breve JCM 1273 | 70.0 | 1.1 | 105.6 | 2.8 | No | NA |
| B. adolescentis JCM 1275 | 50.4 | 0.2 | 87.7 | 0.2 | Yes | + |
| B. animalis BB-12 | 2.5 | 0.4 | 195.4 | 2.5 | Yes | + |
| B. scardovii BAA-773 | 1.6 | 3.2 | 0.5 | 8.2 | Yes | + |
| L. paracasei JCM 1163 | 6.3 | 4.6 | 1.5 | 5.3 | No | NA |
| L. plantarum WCFS1 | 12.7 | 2.4 | 0.0 | 3.2 | Yes | + |
| L. plantarum 299v | 0.2 | 2.1 | 0.0 | 3.7 | No | NA |
| L. brevis KABP-052 (CECT | 0.0 | 0.3 | 0.1 | 0.3 | Yes | − |
| 7480) | | | | | | |
| L. rhamnosus GG | 1.3 | 1.5 | 0.4 | 3.7 | Yes | − |
| L. reuteri DSM 17938 | 0.7 | 4.6 | 1.5 | 5.3 | No | NA |
| S. boulardii CNCM I-754 | 0.4 | 1.2 | 0.6 | 3.9 | No | NA |

ppk, polyphosphate kinase gene; NA, Non-applicable.

In addition, the presence of ppk gene was assessed among the available genomes of the strains under study by BLAST (TABLES 2 and 3). Consistent with phenotypic results, ppk sequence was found in all tested Bifidobacteria and in some Lactobacilli genomes. However, given the differences in polyP production between strains, data supports that regulation mechanisms are different between strains. In fact, polyP biosynthesis in bacteria appears to be regulated on post-transcriptional and/or post-translational level.

TABLE 3
Identification of ppk gene in the available in genomes by BLAST.

ppk

| | Reference | Identity | Coverage |
|---|---|---|---|
| Strains | Genome | (%) | (%) |
| B. longum subsp. longum KABP-042 | AE014295.3 | 99 | 100 |
| (CECT 7894) | | | |
| B. longum subsp. longum 36524 ™ | AE014295.3 | 99 | 100 |
| B. longum subsp. longum ATCC 15707 | AE014295.3 | 98 | 100 |
| B. breve ABP734 | AE014295.3 | 93 | 100 |
| B. animalis BB-12 | AE014295.3 | 82 | 100 |
| B. adolescentis JCM 1275 | AE014295.3 | 85 | 98 |
| B. scardovii BAA-773 | AE014295.3 | 85 | 100 |
| L. plantarum WCFS1 | AL935263.2 | 100 | 100 |
| L. brevis KABP-052 (CECT 7480) | AL935263.2 | ND | ND |
| L. rhamnosus GG | AL935263.2 | ND | ND |

ND: not detected.

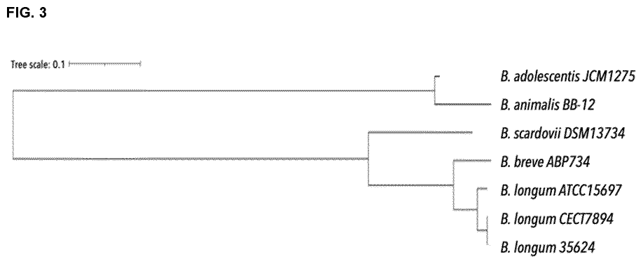

Given the differences observed between ppk sequences in bifidobacterial strains, their aminoacidic sequences were aligned and a tree was constructed. Results showed that bifidobacterial PPK can be grouped in two clades (FIG. 3), one comprises B. animalis and B. adolescentis strains and the other comprises B. scardovii, B. longum and B. breve strains.

### Example 2: Stability of Bifidobacterium longum Subsp. Longum KABP-042 (CECT 7894) in Final Product

The stability of probiotic products depends on several factors including industrial processes of manufacturing and storing and intrinsic characteristic of the probiotic strains.

The industrial processes have been optimized to reduce the loss of viability of strains during production and storage. In addition, manufacturers tend to begin with higher doses of probiotic bacteria to counteract loss during product shelf-life. However, the natural reduced aerotolerance of Bifidobacteria makes more difficult the maintenance of stability during product shelf-life extension compared to other probiotic species.

In this study, the stability of B. longum subsp. longum KABP-042 (CECT 7894) in final product was studied.

### 2.1 Materials and Methods

Final product of B. longum subsp. longum KABP-042 (CECT 7894) was formulated in a matrix containing the active ingredient (minimum 109 colony forming units, cfus), sunflower oil (up to 10 mL) and DL-Apha tocopherol (4 mg). The product was packaged in glass amber bottles and stored under Zone II conditions (25° C., 60% Relative Humidity (RH)).

The quantity of active ingredient (probiotic strain) was chosen to meet recommended cfu/dose following available guidelines.

The stability of the strain was studied by measuring cfus by plate counting according to ISO 29981 at 0, 1, 3 and 6 months after production. Results were expressed in LOG (cfus). Trend line was obtained and predicted cfus at 12 months were estimated. Fold and log reduction comparing cfus between 0 and 12 months were calculated.

### 2.2 Results

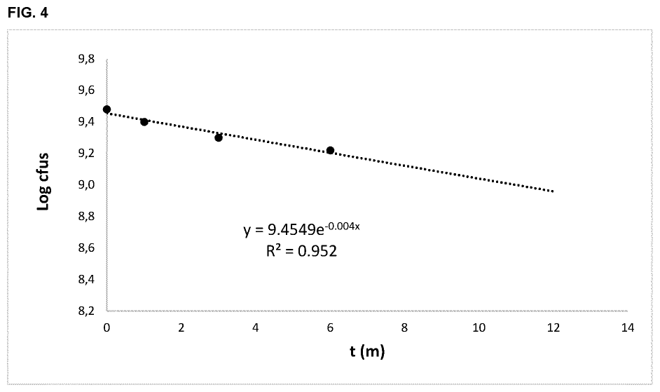

FIG. 4 indicates live B. longum subsp. longum KABP-042 (CECT 7894) found in final product over time (0-6 months) and predicted trend line. At 12 months, LOG (cfus) was estimated at 9.01. This outcome revealed a 3-fold reduction over 12 months (i.e., a ˜0.5 LOG loss), indicating a good stability of the product. Therefore, a 3× overdose at manufacturing would be enough to ensure 109 cfus of live bacteria at 12 months.

### Example 3: Additional Probiotic Characteristics of Bifidobacterium longum Subsp. longum KABP-042 (CECT 7894)

### 3.1 Materials and Methods

Characterization of B. longum subsp. longum KABP-042 (CECT 7894) capacity to resist gastrointestinal conditions, adhere to intestinal epithelium and utilize Human Milk Oligosaccharides (HMOs) was performed. L. rhamnosus GG (ATCC 53103) and B. longum subsp. longum ATCC 15707 were used as controls as indicated. Lactobacilli strains were routinely grown in MRS at 37° C. in anaerobiosis. Bifidobacteria strains were grown in the same conditions except MRS was supplemented with 0.1% (w/v) Cysteine-HCl (MRScys).

Gastric stress resistance and bile salt survival was studied by exposing the strains to simulated gastric solutions (per L: NaCl 7.3 g, KCl 0.52 g, NaHCO₃3.78 g and pepsin 3 g) at pH 2.3 for 30 min and at pH 3 for 90 min, and culture medium containing 0.3% (w/v) bile salts for 180 min. Proliferative bacteria were counted by serial dilution and counting method before and after incubation times. Commercial probiotic strain L. rhamnosus GG was used as reference.

Adhesion to the intestinal epithelium was studied in vitro using Caco-2 intestinal epithelial cells. Bacterial suspensions were added to Caco-2 monolayers (Multiplicity of infection (MOI) 1:5 cells to probiotic) and to wells without Caco-2 cells as controls. After 1 h of incubation at 37° C., medium was removed, cells were detached, and suspensions recovered. Bacteria were enumerated in the obtained suspension by serial dilutions and plate count. Bacteria in the medium of the control wells were also quantified. B. longum subsp. longum ATCC 15707 was used as quality control with a known adhesion percentage of 47-55.

HMOs degradation capacity was tested by growing the strain in MRS with the HMO Lacto-N-tetraose (1%) as unique carbon source. MRS with glucose 1% was used as positive control. MRS without carbon source was used as negative control. Growth was monitored for 24 h.

B. longum subsp. longum KABP-042 (CECT 7894) genome sequence was obtained by Illumina Hiseq, reads were assembled and annotated. Genes of interest such as adhesins, bacteriocins, HMO-degrading enzymes and bile salts hydrolases were searched in the genome by BLAST.

### 3.2 Results

Resistance to the gastric condition was assessed by simulating a fast-gastric passage with no pH buffering (pH 2.3 for 30 min) and a slow postprandial digestion with pH buffering (pH 3 for 90 min). B. longum subsp. longum KABP-042 (CECT 7894) as well as the well-known probiotic strain L. rhamnosus GG showed a loss <1 log cfu/mL in gastric challenges at pH 2.3 and pH 3 (TABLE 4). In addition, B. longum subsp. longum KABP-042 (CECT 7894) showed a high tolerance to bile salts with a loss <0.5 log cfu/mL at similar level to L. rhamnosus GG. Furthermore, one copy of the bsh gene-encoding for a bile salt hydrolase enzyme—was found in B. longum subsp. longum KABP-042 (CECT 7894) genome, confirming the strain is well adapted to the gastrointestinal tract.

TABLE 4
Resistance to gastric stress and bile salts and adhesion to intestinal epitehlium. Values
presented are means and standard deviations of LOG cfu/mL or % of LOG cfu/mL. L. rhamnosus
GG and B. longum subsp. longum ATCC 15707 were used as controls.

| | Gastric solution at pH 2.3 | | | Gastric solution at pH 3 | | | MRS + bile salts 0.3% | | | |
|---|---|---|---|---|---|---|---|---|---|---|
| | | | Loss | | | Loss | | | Loss | |
| | | | t = 0 | | | t = 0 | | | t = 0 | Adhesion |
| | Count | | min − | Count | | min − | Count | | min − | to intestinal |
| | t = 0 | t = 30 | t = 30 | t = 0 | t = 90 | t = 90 | t = 0 | t = 90 | t = 90 | epithelium |
| Strain | min | min | min | min | min | min | min | min | min | (%) |
| B. longum | 6.25 ± | 5.67 ± | 0.58 ± | 6.81 ± | 6.53 ± | 0.28 ± | 6.79 ± | 6.57 ± | 0.22 ± | 70.8 ± |
| subsp. longum | 0.14 | 0.07 | 0.07 | 0.69 | 0.62 | 0.06 | 0.22 | 0.27 | 0.09 | 1.20 |
| KABP042 | | | | | | | | | | |
| (CECT 7894) | | | | | | | | | | |
| L. rhamnosus | 6.98 ± | 6.19 ± | 0.68 ± | 6.92 ± | 7.09 ± | − 0.17 ± | 7.01 ± | 6.85 ± | 0.15 ± | NA |
| GG | 0.23 | 0.21 | 0.08 | 0.24 | 0.19 | 0.16 | 0.42 | 0.26 | 0.24 | |
| B. longum | NA | NA | NA | NA | NA | NA | NA | NA | NA | 51.2 ± |
| subsp. longum | | | | | | | | | | 1.00 |
| ATCC 15707 | | | | | | | | | | |

NA, non-applicable.

B. longum subsp. longum KABP-042 (CECT 7894) was confirmed to adhere to the intestinal epithelium with a 70.8% adhesion capacity (TABLE 4). The strain adhered greater than the moderately adherent control strain B. longum subsp. longum ATCC 15707 (51.2%). Genome analyses confirmed the strain is well equipped with several adhesion proteins and domains. Adhesion of bacteria to human tissues is a prerequisite for an effective bacterial colonization, which is in turn a desirable trait to achieve a persistent health benefit effect.

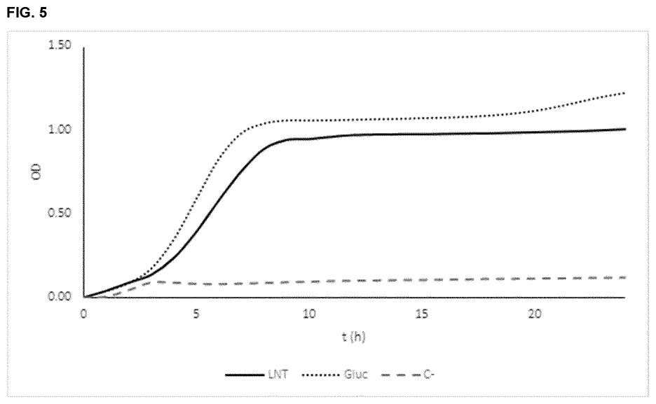

B. longum subsp. longum KABP-042 (CECT 7894) was able to grow in presence of the HMO Lacto-N-Tetraose (LNT) as the sole carbon source (FIG. 5). The genome was confirmed to harbor HMO-degrading genes including lacto-N-biosidase, beta-galactosidase, alpha-galactosidase, hexosaminidase, beta-glucuronidase. Thus, HMOs utilization by B. longum subsp. longum KABP-042 (CECT 7894) was confirmed phenotypically and genotypically, proving it is well adapted to the infant intestine.

In addition, B. longum subsp. longum KABP-042 (CECT 7894) genome harbors other genes encoding Carbohydrate Active Enzymes (CAZy), suggesting its ability to degrade a wide range of complex substrates, such as those coming from a varied human diet. B. longum subsp. longum KABP-042 (CECT 7894) appears to have a versatile carbohydrate metabolism.

Further analysis showed the presence of genes encoding Lanthipeptide B, serpin and adhesins. Lanthipeptide B (Lantibiotic) is a class-I bacteriocin produced by B. longum strains that exhibits strong antimicrobial activity against a range of gram-negative and gram-positive pathogenic bacteria. Serpins (from Serine Protease Inhibitors) selectively inactivates human neutrophil and pancreatic elastases (proteases), resulting in anti-inflammatory effect and contributing to maintaining gut homeostasis.

Overall, in vitro and in silico analysis of B. longum subsp. longum KABP-042 (CECT 7894) confirms the probiotic characteristics of the strain indicating it is well adapted to the human gastrointestinal tract including the infant gut since it has the capacity to degrade HMOs.

### Example 4: Effect of B. longum CECT7894-Derived polyP in the Protection of the Intestinal Barrier

Postbiotic effect of polyP is related to its role in maintain intestinal homeostasis and protecting intestinal barrier function. One mechanism of action is the induction of the cytoprotective factor heat shock protein HSP27 in the intestinal cell (Alcántara et al., 2018).

It was studied whether polyP produced by B. longum CECT 7894 has effect on barrier integrity and gut permeability. In addition, it was explored if the effect is related to HSP27 production or the induction other markers of barrier integrity including tight junction proteins.

### 4.1. Materials and Methods

4.1.1 Preparation of B. longum CECT 7894 Samples and Quantification of polyP Production

B. longum CECT 7894 was grown in MEI medium and Low Phosphate (LP) medium. The last medium has the same composition as MEI but without the addition of polyP precursors (K₂HPO₄ and KH₂PO₄), thus the strain cannot produce high amounts of polyP. After 16 h growth cultures were centrifuged and supernatants collected, filtered, and adjusted to neutral pH. PolyP amounts were measured as described in EXAMPLE 1.

### 4.1.2. Evaluation of Barrier Integrity and Permeability

Integrity of Caco-2 cells monolayer was evaluated by measuring transepithelial electrical resistance (TEER) and permeability by the apparent permeability coefficients (Papp) of the paracellular transport marker Lucifer Yellow.

Caco-2 cells were seed in porous membrane inserts with apical (upper) and basolateral (lower) compartments. Medium Essential Medium Eagle (MEM) was added to both compartments. Cells were treated with supernatants of B. longum CECT 7894 grown in MEI and LP media. Additional cells were treated with MEM, non-fermented MEI and LP media and used as controls.

After 72 h of treatment, TEER and permeability were determined. TEER was measured with a Millicell®-ERS voltammeter. For the permeability assay, Lucifer Yellow was added to the apical compartment. At 15, 30, 45, 60, 90 and 120 min, aliquots were taken from the basolateral compartment and the fluorescence of the Lucifer Yellow transported was measured with a fluorescence microplate reader at excitation/emission wavelengths of 485/520 nm.

### 4.1.3. Quantification of HSP27 Production

The production of HSP27 was studied in Caco-2 intestinal epithelial cells in confluence by Western blot assay as described by Alcántara et al., 2018 with some modifications. Bacterial supernatants were added to the cell cultures and incubation proceeded for 16 h. MEI and LP media were used as controls. To recover HSP27, cells were lysed with SDS-PAGE and boiled for 5 min. Proteins were separated in SDS-PAGE gel and then transferred to a nylon membrane (blot). The blots were incubated with a rabbit polyclonal anti-HSP27 serum or with a mouse monoclonal anti-β-actin antibody (protein used for normalization). After washing, secondary antibodies peroxidase-conjugated anti-rabbit IgG and anti-mouse IgG, respectively, were used. Blots images were captured, and proteins were quantified in an Imagin 680 system.

### 4.1.4. Expression of Genes Encoding Tight Junction Proteins

Caco-2 cells were exposed for 16 h to supernatants of B. longum CECT 7894 grown in MEI and LP media. Then, cells were recovered, and RNA extracted with TRIZOL reagent. cDNA was obtained from RNA using SuperScript VILO cDNA synthesis kit. Quantitative PCR (qPCR) reactions were performed with SYBR Green in the conditions indicated by manufacturer. Expression of tight junction proteins Zonula ocludens-1 (ZO1), Junctional adhesion protein-1 (JAM1) and occluding was quantified. Expression of 18S rRNA and GADPH genes were used for normalization.

### 4.2 Results

First, polyP amounts in supernatants grown in MEI medium were higher than the amounts in supernatants grown in LP medium (TABLE 5). Of note, amounts in MEI were lower than those quantified in EXAMPLE 1 in the same medium. However, in EXAMPLE 1 polyP is measured intracellularly while in EXAMPLE 4 polyP is measured extracellularly. Extracellular production was studied here to mimic the conditions in the gut, i.e., the extracellular polyP in contact with gut barrier.

TABLE 5
PolyP amount (nmol) in B. longum CECT 7894 supernatants grown
under high (MEI medium) or low (LP medium) phosphate conditions for
16 h. Growth (OD₅₅₀) in each condition is indicated.

| Medium | PolyP | OD |
|---|---|---|
| MEI | 2.24 | 3.2 |
| LP | 0.22 | 1.1 |

Experiments of Caco-2 monolayers in bicompartmental system showed that apical exposure to supernatants of B. longum CECT 7894 with high concentration of polyP (i.e., from cultures in MEI medium) displayed higher TEER (indicative of a greater resistance of the cell barrier) compared to supernatants with low polyP amounts and controls. Experiment measuring by the flow of Lucifer Yellow from apical to basolateral compartment also indicated that a high polyP concentration derived from B. longum CECT 7894 significantly reduced the permeability of the compound compared with low polyP supernatant and controls (shown in FIG. 6). These results indicate polyP produced by B. longum CECT 7894 promote a stronger functional barrier preventing intestinal permeability. Importantly, the effects were significant even though the amounts of polyP in the supernatants were lower than intracellular, suggesting little amounts of polyP produced by B. longum CECT 7894 are enough to have a beneficial effect in the barrier integrity.

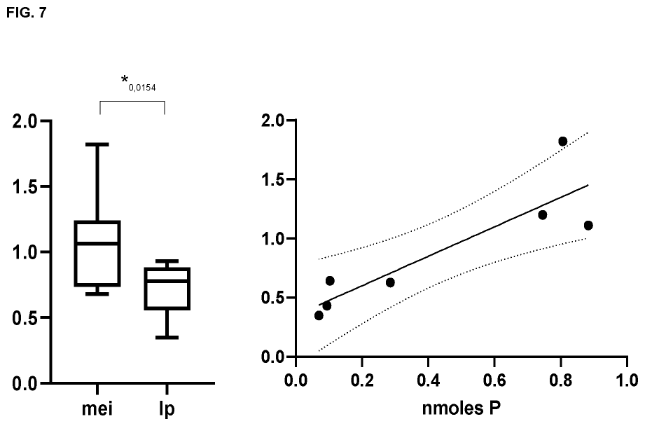

Western blot analysis of HSP27 production in intestinal epithelial cells showed that supernatants with high concentration of polyP produced by B. longum CECT 7894 (i.e., from cultures in MEI medium) induced a significantly higher production of HSP27 compared to the supernatants from cultures with low concentration of polyP (i.e., from cultures in LP medium). In addition, a correlation was also observed between HSP27 expression and polyP concentrations in supernatants of B. longum CECT 7894 using different samples with different amounts of polyP (shown in FIG. 7). These outcomes indicate that B. longum CECT 7894 can affect HSP27 production through the synthesis of polyP and therefore the strain has a protecting effect of the intestinal epithelium.

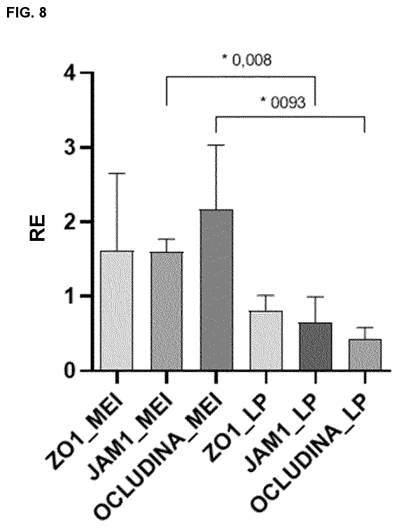

Furthermore, the expression of tight junction proteins ZO1, JAM1 and Occludin, which are crucial for the maintenance of barrier integrity, was induced by the presence of high polyP amounts in the supernatant of B. longum CECT 7894 as compared to low polyP supernatants (shown in FIG. 8).

Overall, these outcomes confirm the strain B. longum CECT 7894 through the production of polyP is able to enhance the barrier integrity reducing intestinal permeability by the induction of the production of cytoprotective protein HSP27 and tight junction proteins. Therefore, B. longum CECT 7894 has a positive effect in gut barrier homeostasis.

### Example 5: Effect of Breast Milk, the HMO Lacto-N-Tetraose and Polyamines in the PolyP Production Capacity of B. longum CECT7894

B. longum is naturally found in human breast milk and in the intestine of infants. Human milk contains amounts of phosphate (the substrate of polyP). It was studied whether B. longum CECT 7894 is able to produce polyP in presence of breast milk. In addition, some evidence in other bacteria has suggested polyamines and carbon source can affect polyP metabolism (Anand et al., 2019). Since breast milk contains polyamines and carbohydrates HMOs, it was tested if polyamines and the HMO Lacto-N-tetraose (LNT), which B. longum CECT 7894 utilizes (as confirmed in EXAMPLE 3), can affect polyP biosynthesis in the studied strain.

### 5.1. Materials and Methods

B. longum CECT 7894 was grown in medium MEI without glucose supplemented with i) breast milk (1% v/v); ii) LNT (1% w/v); iii) polyamines, in quantities found in breast milk: 70.0, 424.2 and 610.0 10 nmol/dl of putrescine, spermidine and spermine, respectively, and glucose (0.5% w/v); and iv) glucose (0.5% w/v) as positive control. Growth (OD550) and polyP production were determined after 6 and 16 h of incubation.

### 5.2 Results

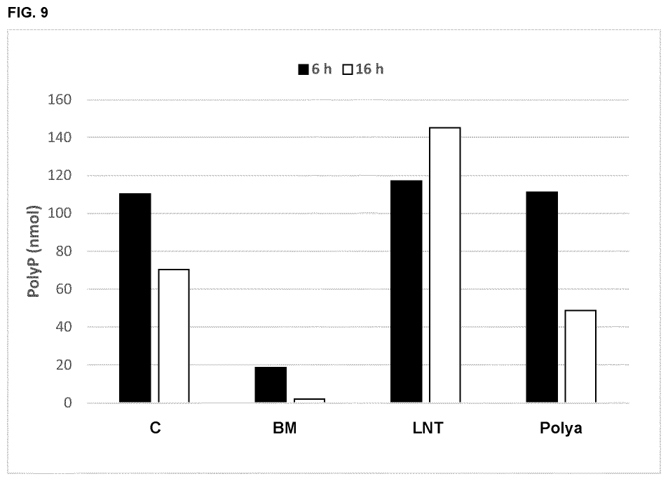

Analysis of B. longum CECT 7894 growth in presence of breast milk (with the sugars present in breast milk as the unique carbon source) showed that despite the strain only reach a low OD, it is still able to produce some amounts of PolyP at 6 h. Growth with LNT as unique carbon source was lower than the growth in control condition at 6 h (OD 1.8 vs 2.9). However, the strain produced a greater amount of polyP (117.0 vs 110.2). In addition, polyP remained for a longer period in LNT compared to control (145.0 vs 70.2 at 16 h). The presence of polyamines in MEI medium with glucose did not affect growth nor polyP production (see TABLE 6 and FIG. 9).

TABLE 6
PolyP quantification (nmol) and growth (OD₅₅₀) of B. longum CECT
7894 cultures incubated under different conditions at 6 and 16 h.

| | |
|---|---|
| 6 h | 16 h |

| Condition | PolyP | OD | PolyP | OD |
|---|---|---|---|---|
| Control | 110.2 | 2.9 | 70.2 | 2.7 |
| (glucose) | | | | |
| Breast milk | 18.5 | 0.5 | 2.0 | 0.5 |
| LNT | 117.0 | 1.8 | 145.0 | 2.9 |
| Polyamines | 111.0 | 3.0 | 48.6 | 2.7 |

In conclusion, these results indicate B. longum CECT 7894 can produce polyP in presence of breast milk and the HMO LNT enhances the biosynthesis of polyP, suggesting a LNT-dependent regulation of polyP metabolism in the studied strain. Importantly, this is the first time an interaction of an HMO and polyP is showed and highlights the beneficial role that B. longum CECT 7894 supplementation can have in e.g., infants.

### Example 6: Cross-Feeding of B. longum CECT 7894 with Bifidobacteria that Utilizes 2FL

It was studied whether B. longum CECT 7894 was able to growth with the HMO 2′-FL, through crossfeeding of other Bifidobacteria present in e.g., human milk or the human gut.

### 6.1. Materials and Methods

B. bifidum Bb01 (CECT 30646) was grown in MRS medium with 2′-Fucosyl-lactose (2′-FL) (4% w/v) as unique carbon source for 48 h. Supernatant was recovered and filtered to remove cells. Supernatant was mixed with fresh medium MRS without carbon source (1:1). B. longum CECT 7894 was grown in the mixture for 24 h and OD was monitored.

### 6.2. Results

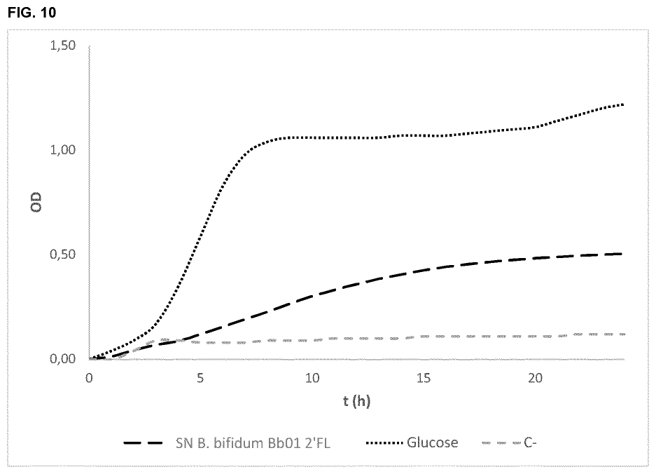

B. longum CECT 7894 was able to grow in presence of the supernatant of B. bifidum Bb01 (CECT 30646) cultured with 2′-FL reaching an OD of 0.5 (FIG. 10). This outcome demonstrates B. longum CECT 7894 can be feed by other Bifidobacteria that utilizes 2′-FL. Thus, together with results of EXAMPLE 3 (FIG. 5), B. longum CECT 7894 is able to growth in presence of the two most abundant HMOs in breast milk (LNT and 2′-FL).

### REFERENCES

### Non-Patent Literature

Qian, Y., Borowski, W. J., & Calhoon, W. D. (2011). Intracellular granule formation in response to oxidative stress in Bifidobacterium. International Journal of Food Microbiology, 145(1), 320-325. https://doi.org/10.1016/j.ijfoodmicro.2010.11.026
Anand, A., Sato, M., & Aoyagi, H. (2019). Screening of phosphate-accumulating probiotics for potential use in chronic kidney disorder. Food Science and Technology Research, 25(1), 89-96. https://doi.org/10.3136/fstr.25.89
Saiki, A., Ishida, Y., Segawa, S., Hirota, R., Nakamura, T., & Kuroda, A. (2016) A lactobacillus mutant capable of accumulating long-chain polyphosphates that enhance intestinal barrier function. Bioscience, Biotechnology, and Biochemistry, 80(5), 955-961. https://doi.org/10.1080/09168451.2015.1135041
Alcántara, C., Blasco, A., Zúñiga, M., & Monedero, V. (2014) Accumulation of Polyphosphate in lactobacillus spp. and its involvement in stress resistance. Applied and Environmental Microbiology, 80(5), 1650-1659. https://doi.org/10.1128/aem.03997-13
Alcántara, C., Coll-Marqués, J. M., Jadán-Piedra, C., Vélez, D., Devesa, V., Zúñiga, M., & Monedero, V. (2018) Polyphosphate in lactobacillus and its link to stress tolerance and probiotic properties. Frontiers in Microbiology, 9. https://doi.org/10.3389/fmicb.2018.01944
Perez, M., Astó, E., Huedo, P., Alcántara, C., Buj, D., & Espadaler, J. (2020) Derived Postbiotics of a multi-strain probiotic formula clinically validated for the treatment of irritable bowel syndrome. The FASEB Journal, 34(S1), 1-1. https://doi.org/10.1096/fasebj.2020.34.s1.05062
Abstract Xiao, F., Dong, F., Li, X., Li, Y., Yu, G., Liu, Z., Wang, Y., Zhang, T. (accepted 30 May 2022). Bifidobacterium longum CECT 7894 improves the efficacy of infliximab for DSS-induced colitis via regulating the gut microbiota and bile acid metabolism. Front. Pharmacol. Sec. Gastrointestinal and Hepatic Pharmacology doi: 10.3389/fphar.2022.902337
Shah, ED., Farida, JP., Siegel, CA., Chong, K., Melmed, GY. (2017) Risk for overall infection with anti-TNF and anti-integrin agents used in IBD: a systematic review and meta-analysis. Inflamm Bowel Dis 2017 April; 23(4):570-577. doi: 10.1097/MIB.0000000000001049
Bischoff, SC., Barbara, G., Buurman, W., Ockhuizen T., Schulzke, JD., Serino, M., Tilg, H., Watson, A., Wells, J M. (2014) Intestinal permeability—a new target for disease prevention and therapy. BMC Gastroenterol. 2014; 14: 189. doi: 10.1186/s12876-014-0189-7
Segawa S., Fujiya M., Konishi H., Ueno N., Kobayashi N., Shigyo T., Kohgo Y. (2011) Probiotic-derived polyphosphate enhances the epithelial barrier function and maintains intestinal homeostasis through integrin-p38 MAPK pathway. PLOS One 2011; 6(8):e23278 doi: 10.1371/journal.pone.0023278
Tanaka, K., Fujiya, M., Konishi, H., Ueno, N., Kashima, S., Sasajima, J., Moriichi, K., Ikuta, K., Tanabe, H., Kohgo, Y. (2015) Probiotic-derived polyphosphate improves the intestinal barrier function through the caveolin-dependent endocytic pathway. Biochemical and Biophysical Research Communications, vol. 467, Issue 3, 20 Nov. 2015 pages 541-548. http://dx.doi.org/10.1016/j.bbrc.2015.09.159
Fujiya, M., Ueno, N., Hashima, S., Tanaka, K., Sakatani, A., et al., (2020) Long-Chain Polyphosphate Is a Potential Agent for Inducing Mucosal Healing of the Colon in Ulcerative Colitis. Clinical Pharmacology & Therapeutics vol. 197, Issue 1, February 2020, pages 452-461 doi: 10.1002/cpt. 1628
Kelly, JR., Kennedy, PJ., Cryan, J F., Dinan, TG., Clarke, G., Hyland NP. (2015) Breaking down the barriers: the gut microbiome, intestinal permeability and stress-related psychiatric disorders. Front Cell Neurosci. 2015; 9: 392. doi: 10.3389/fncel.2015.00392
Verhaar, BJH., Prodan, A., Nieuwdorp, M., Muller, M. (2020) Gut microbiota in Hypertension and Atherosclerosis: a review. Nutrients 2020 Sep. 29; 12(10):2982. doi: 10.3390/nu12102982
Rogler, G., Rosano, G. (2014) The heart and the gut. European Heart Journal 35, 426-430 doi: 10.1093/eurheartj/eht271
Jiang, C., Li, G., Huang, P., Liu, Z., Zhao, B. (2017) The gut microbiota and Alzheimer's disease. J Alzheimers Dis 2017; 58(1):1-15. doi: 10.3233/JAD-161141.
Cox, AJ., West, NP, Cripps, A W. (2015) Obesity, inflammation, and the gut microbiota. Lancet Diabetes Endocrinol. 2015 March; 3(3):207-15. doi: 10.1016/S2213-8587(14)70134-2
Pike, MG., Heddle, RJ., Boulton, P., Turner, MW., Atherton, DJ. (1986) Increased intestinal permeability in atopic eczema. J Invest Dermatol 1986 February; 86(2):101-4. doi: 10.1111/1523-1747.ep12284035
Tajik, N., Frech, M., Schulz, O. et al. (2020) Targeting zonulin and intestinal epithelial barrier function to prevent onset of arthritis. Nat Commun 11, 1995 (2020). https://doi.org/10.1038/s41467-020-15831-7
Massier L, Blüher M, Kovacs P and Chakaroun RM (2021) Impaired Intestinal Barrier and Tissue Bacteria: Pathomechanisms for Metabolic Diseases. Front. Endocrinol. 12:616506. doi: 10.3389/fendo.2021.616506

### Patent Literature

WO2015018883A2
JP2006176450A

1. A probiotic composition comprising:
Bifidobacterium longum subsp. longum strain deposited under the Budapest Treaty in the Spanish Type Culture Collection, CECT, under accession number CECT 7894, or a bacterial strain derived thereof, for use in the treatment of non-intestinal conditions associated with increased intestinal permeability in a subject,
wherein the treatment is by producing polyphosphate, wherein the derived bacterial strain:
(a) has a genome with at least 99% average nucleotide identity, ANI, to the genome of the correspondent deposited strain; and
(b) retains the ability of the correspondent deposited strain to produce polyphosphate.

2. The probiotic composition for use according to claim 1, wherein the non-intestinal condition is an immune disorder or disease, a metabolic or cardiovascular disorder or disease, or a neurological or psychiatric disorder or disease.

3. The probiotic composition for use according to claim 2, wherein the metabolic or cardiovascular disorder or disease is selected from the group consisting of obesity, diabetes, insulin resistance, non-alcoholic fatty liver disease, liver cirrhosis, atherosclerosis, hypertension, chronic heart failure, and stroke; the immune disorder or disease is selected from the group consisting of non-alimentary allergy/hypersensitivity, immunosenescence, multiple sclerosis, rheumatoid arthritis, lupus erythematosus, sarcopenia, asthma, allergic rhinoconjunctivitis, and atopic dermatitis; and the neurological or psychiatric disorder or disease is selected from the group consisting of Alzheimer's disease, autistic spectrum disorders, schizophrenia and depression.

4. The probiotic composition for use according to claim 1, further comprising at least one human milk oligosaccharide.

5. The probiotic composition for use according to claim 1, wherein the non-intestinal conditions associated to increased intestinal permeability are related to pre-term birth, ageing, high-intensity physical activity, dietary imbalances, infection, drug treatment and/or stress.

6. The probiotic composition for use according to claim 1, wherein the subject is a human, and the human is selected from the group consisting of elderly people, pre-term infants, infants, athletes, and fragile people.

7. The probiotic composition for use according to claim 6, wherein the infant is selected from the group consisting of a pre-term infant, a fragile infant, an infant born with a subnormal birth weight, an infant subject of intrauterine growth retardation, an infant born by C-section, an infant administered with antibiotics, a formula-fed infant and a breast-fed infant.

8. The probiotic composition for use according to claim 1, wherein the derived bacterial strain has a genome with at least 99.5% average nucleotide identity to the genome of the correspondent deposited strain.

9. The probiotic composition for use according to claim 1, wherein the production of polyphosphate of the strain Bifidobacterium longum subsp. longum CECT 7894 or a bacterial strain derived thereof is higher than the production of polyphosphate of a control strain, when the polyphosphate production is determined at 6 h and/or 16 h of culture by the following steps:
(a) culturing the strains inoculated at OD 0.1 in malic enzyme induction medium containing per liter, w/v: 0.5% yeast extract, 0.5% tryptone, 0.4% K₂HPO₄, 0.5% KH₂PO₄, 0.02% MgSO₄·7H₂O, 0.005% MnSO₄, 1 ml of Tween 80, 0.05% cysteine, and 0.5% glucose, at 37° C. and under anaerobic conditions;
(b) harvesting cells by centrifugation and lysis in 1 ml of 5% sodium hypochlorite with gentle agitation for 45 min at room temperature;
(c) centrifugating the insoluble material at 16,000 g for 5 min at 4° C. to obtain a pellet and washing twice with 1 ml of 1.5 M NaCl plus 1 mM EDTA at 16,000 g for 5 min at 4° C.;
(d) extracting polyphosphate from the pellets with two consecutive washes with 1 ml of water and centrifugating at 16,000 g for 5 min at 4° C. between them;
(e) precipitating polyphosphate in the pooled water extracts by adding 0.1 M NaCl and 1 volume of ethanol, followed by incubation on ice for 1 h;
(f) centrifugating at 16,000 g for 10 min and resuspending the polyphosphate pellet in 50 μL of water;
(g) building a standard curve, relating polyphosphate-derived phosphate amount to fluorescence intensity, following the steps:
i. hydrolyzing serial dilutions of a sample of polyphosphate isolated from the control strain Lactobacillus plantarum WCFS1 with a volume of 2 M HCl and incubation at 95° C. for 15 min;
ii. neutralizing the dilutions by adding half volume of 2 M NaOH;
iii. measuring the released phosphate with BIOMOL Green Kit to obtain the amount of phosphate in each dilution;
iv. measuring the released phosphate by fluorescence using the 4′,6-diamidino-2-phenylindole, DAPI, at a final concentration of 10 μM in 50 mM Tris-HCl PH 7.5, 50 mM NaCl buffer with an excitation wavelength of 415 nm and emission at 550 nm in a fluorimeter to obtain the fluorescence value in each dilution; and
v. building the standard curve with phosphate values obtained in (iii) and the corresponding fluorescence values obtained in (iv); and
(h) quantifying polyphosphate from the resuspended fractions of step (f):
1) measuring phosphate by fluorescence using DAPI at a final concentration of 10 μM in 50 mM Tris-HCl pH 7.5, 50 mM NaCl buffer with an excitation wavelength of 415 nm and emission at 550 nm in a fluorimeter;
2) calculating the amount of polyphosphate by means of the standard curve; and
3) expressing polyphosphate value in nmol of phosphate.

10. The probiotic composition for use according to claim 9, wherein the production of polyphosphate of B. longum subsp. longum CECT 7894 or a bacterial strain derived thereof at 6 h is at least 10-fold higher and at 16 h is higher than the production of polyphosphate of the control strain L. plantarum WCFS1, wherein the production of polyphosphate of the control strain L. plantarum WCFS1 and the levels of polyphosphate by the control strain at 16 h is non-existent.

11. The probiotic composition for use according to claim 1, comprising Bifidobacterium longum subsp. longum strain deposited under the accession number CECT 7894.

12. A combination comprising:
(i) a probiotic composition comprising:
Bifidobacterium longum subsp. longum strain deposited under the Budapest Treaty in the Spanish Type Culture Collection, CECT, under accession number CECT 7894, or a bacterial strain derived thereof, wherein the derived bacterial strain:
(a) has a genome with at least 99% average nucleotide identity, ANI, to the genome of the correspondent deposited strain; and
(b) retains the ability of the correspondent deposited strain to produce polyphosphate; and
(ii) at least one human milk oligosaccharide,
wherein the combination is configured for simultaneous, separate or sequential administration.

13. The combination according to claim 12, wherein the human milk oligosaccharide is selected from the group consisting of a fucosylated oligosaccharide, a sialylated oligosaccharide, a N-acetyl-lactosamine and a combination thereof.

14. The combination according to claim 13, which comprises 2′-fucosyllactose and/or lacto-N-tetraose.

15. The combination according to claim 1, further comprising a Bifidobacterium bifidum strain, particularly B. bifidum CECT 30646.

16. The combination according to claim 1, for use in the treatment of conditions associated to increased intestinal permeability in a subject, wherein the treatment is by producing polyphosphate, and wherein the condition is selected from the group consisting of an immune disorder or disease, a metabolic or cardiovascular disorder or disease, a neurological or psychiatric disorder or disease and a gastrointestinal disorder or disease.
